SEQUENCE LISTING (72 sequences)

SEQ ID NO: 1 - DNA, 191 bp, human chromosome X genomic contig NT_025307.13 Str #1
tcattaatatgaactcttgttcaaaggaaatatatatatatatatatatacacacacacacacacacacacacatatatacacacacacacacatacacacacatatatacacacacacacacatatatacacacacacacagaaaccctatatatatatatctcagagctgagaaaggtatgtgtatatg

SEQ ID NO: 2 - DNA, 165 bp, human chromosome X genomic contig NT_025307.13 Str#2
gataaaacataagtaaagacgaaggaattcgaataaaacataataaataaataacaataataaaccagaacacacacacacacacacacacacacacacacacacacacacacacacacacagaacgttcattttctttaaagcaataatatacttggccacaaa

SEQ ID NO: 3 - DNA, 146 bp, human chromosome X genomic contig NT_025307.13 Str#3
ttcattttgcgtgtcatttcacccagtgtgtgtgtgtgtatgtgtgtgtgtgtgtgtgtgtgtgtgtgtagtatgtgcatgtgtcacatttcaggaacattttgttgaaatgaaggatttagaaatttttttggtcccctgctttg

SEQ ID NO: 4 - DNA, 134 bp, human chromosome X genomic contig NT_025307.13 Str#4
cccacaccccctctctaaattaatctatgtgagtcagcccctgcccccccccaccccgctctctctctctctctctctctctctctctctctctctttcttcatttacccccctttcattcttccctggctcac

SEQ ID NO: 5 - DNA, 166 bp, human chromosome X genomic contig NT_025307.13 Str#5
attcatttggtggtgccctacagttggagttcttgtcctagggtgtgtggaataatatgcaaaattcgtgtgtgtgtgtgtgtgtgtgtgtgtgtgtgtgtgtgtgtgtgtagaatttctatggggtctataacctaaaaatattgagaaccctttccctagagtg

SEQ ID NO: 6 - DNA, 187 bp, human chromosome X genomic contig NT_025307.13 Str#6
acccctattcttccctgcacatttatctggggtctctctccctctctctctctctctctctctctctctctctctctctctctctctcctctctctctctctcttctctctctcttctctctctctcatcccctcgttcccttccttgctttcctccccactcctctcaagctctttttgtttccca

SEQ ID NO: 7 - DNA, 130 bp, human chromosome X genomic contig NT_025307.13 str#7
gggagataggaatgatggagtgaattcatcatatgtgatttccacagccaccacacacacacacacacacacacacacacacacacacacgcacacacacactgtatcccccaagatggcccagaaaata

SEQ ID NO: 8 - DNA, 142 bp, human chromosome X genomic contig NT_025307.13 str#8
ttcattctttcttatggctgcatagtattccatggtgtgtgtgtgtgtgtgtgtgtgtgtgtgtgtgtgtgtgtgtataatataaaatcttctttatctaatcatctgtttatgcacacttaggttgattccatgactttgc

SEQ ID NO: 9 - DNA, 188 bp, human chromosome X genomic contig NT_025307.13 Str#9
tagcaatgaggagccactgaaagataagattttaagcaatgctggtgtgtgtgtgtgtgtgtgtgtgtgtgtgtgtgtgtgtgtgtgtgtttgagggagagagagagagagagagagaagagaagagaagaagtcaaattggtattttatagagatactactactactctggccctagtatggagact

SEQ ID NO: 10 - DNA, 132 bp, human chromosome X genomic contig NT_025307.13 Str#10
atctgtttgctgatgacatgatattatatgtagaaaatcctaaagactacacacacacacacacacacacacacacacacacacacacacacacacacaaactattagaataataaatgaattcaggccggg

SEQ ID NO: 11 - DNA, 743 bp, human chromosome X genomic contig NT_025307.13 Str#11
agaaggatccaggtctgctggccatagccgagtgctttgaaagtcaccagtcctgacagcgattcgtgtgtgtgtctgtgtgtgtgtgtgtgtgtgtttatgtgtctgtgtgtgttcgtgtgtgtgtctgtgtgtgtgtttgtgtgtgtatgtctgtgtgtgtgtgtttatgtgtgtgtgtctgtgtgtgtttaagtctgtgtgtgtttgtgtgtgtgtgtctctgtgtgtgtgtctgtatctgtgtgtgtttgtgtctgtgtgtgtttgtgtgtgtgtctgtgtgtgtttgtgtgtgtgtgtctgtgtgtgtgtttatgtgtctgtgtatgtttgtgtgtgtgtttgtgtgtgtgtgtttgtgtttatgtgtgtatgtctgtgtgtgtctgtgtgtctgtgtgtgtatgtgtctgtgtgtgtttatgtgtctgtgtgtgttcgtgtggtgtgtgtgtctgtgtgtgtgtgtttgtgtgtgtatgtctgtgtgtgtgtgtgtttatgtgtgtgtgtctgtgtgtgtttatgtctgtgtgtgtttgtgtgtgtgtgtctcgtgtgtgtgtgtctgtgtgtatctgtgtgtgtttgtgtgtgtgtgtgtctctgtgtgtgtgtgtgtttgtgtatgtttgtgtgtgtgtgtgtgtgtgttgggaatgcccagtctctgcagctgctgaaaggccctgaggcacatgctgtcaggagctggctctgtcctgggcagatatcaccat

SEQ ID NO: 12 - DNA, 208 bp, human chromosome X genomic contig NT_025307.13 SYBL-STR#1b
cgaaatgcttcccttttatccatgaaactatttgagtataaagacgtaagctctagctatgattttgttgtgagtttaaaaaaaagctgtattgtaatggaatactttcacttctcctgtggttttaatactctgagtattacatttctaaattttatgcacacacacacacacgcacacacatactcttctggcaataaagtccctt

SEQ ID NO: 13 - DNA, 167 bp, human chromosome X genomic contig NT_025307.13 SYBL-Str#2b
aatgctgctatgaacatttgtgtacaagtttttgtgtgagcatatatatatattatatattatatatatatatatatgtttttggttctcttgcttacatacctaggaatagaattgctggatcacatggcaactttgtaacattttgaagaagggccaaactttcc

SEQ ID NO: 14 - DNA, 510 bp, human chromosome X genomic contig NT_025307.13 (HSPRY3 promoter region)
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggaggaggagagagaggaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctctgtttctggctccactg

SEQ ID NO: 15 - DNA, 605 bp, human chromosome X genomic contig NT_025307.13 (HSPRY3 coding region) variant 1
atggtgctctgaagggagaagctgagcaatctgcagggcaccctagtgagcacctcttcatctgtgaggaatgtgggcgctgcaagtgcgtcccctgcacagcagctcgccctctcccctcctgctggctgtgcaaccagcgctgcctttgctctgctgagagcctcctcgattatggcacttgtctctgctgtgtcaagggcctcttctaccactgctccactgatgatgaagacaactgtgctgatgagccctgctcttgtgggcctagttcttgctttgtccgctgggcagccatgagcctcatctccctcttcctaccctgcctgtgctgctacctgcctacccgtggatgcctccatctgtgccaacagggctatgatagcctccggcgaccaggctgccgctgcaagaggcacaccaacactgtgtgcagaaagatctcttctggtagtgcacccttccccaaggcccaggaaaagtctgtatgaccttccaacaaggtggatccagagcttttctccttcgagtccccaacagcaaagcataggcctcatctttggagagggggaggagtgataaactagccaaagttagggcctctc

SEQ ID NO: 16 - DNA, 605 bp, human chromosome X genomic contig NT_025307.13 (HSPRY3 coding region variant2)
atggtgctctgaagggagaagctgagcaatctgcagggcaccctagtgagcacctcttcatctgtgaggaatgtgggcgctgcaagtgcgtcccctgcacagcagctcgccctctcccctcctgctggctgtgcaaccagcgctgcctttgctctgctgagagcctcctcgattatggcacttgtctctgctgtgtcaagggcctcttctaccactgctccactgatgatgaagacaactgtgctgatgagccctgctcttgtgggcctagttcttgctttgtccgctgggcagccatgagcctcatctccctcttcctaccctgcctgtgctgctacctgcctacccgtggatgcctccatctgtgccagcagggctatgatagcctccggcgaccaggctgccgctgcaagaggcacaccaacactgtgtgcagaaagatctcttctggtagtgcacccttccccaaggcccaggaaaagtctgtatgaccttccaacaaggtggatccagagcttttctccttctagtccccaacagcaaagcataggcctcatctttggagagggggaggagtgataaactagccaaagttagggcctctc

SEQ ID NO: 17 - DNA, 605 bp, human chromosome X genomic contig NT_025307.13 (HSPRY3 coding region variant3)
atggtgctctgaagggagaagctgagcaatctgcagggcaccctagtgagcacctcttcatctgtgaggaatgtgggcgctgcaagtgcgtcccctgcacagcagctcgccctctcccctcctgctggctgtgcaaccagcgctgcctttgctctgctgagagcctcctcgattatggcacttgtctctgctgtgtcaagggcctcttctaccactgctccactgatgatgaagacaactgtgctgatgagccctgctcttgtgggcctagttcttgctttgtccgctgggcagccatgagcctcatctccctcttcctaccctgcctgtgctgctacctgcctacccgtggatgcctccatctgtgccaacagggctatgatagcctccggcgaccaggctgccgctgcaagaggcacaccaacactgtgtgcagaaagatctcttctggtagtgcacccttccccaaggcccaggaaaagtctgtatgaccttccaacaaggtggatccagagcttttctccttctagtccccaacagcaaagcataggcctcatctttggagagggggaggagtgataaactagccaaagttagggcctctc

SEQ ID NO: 18 - DNA, 605 bp, human chromosome X genomic contig NT_025307.13(HSPRY3 coding region variant4)
atggtgctctgaagggagaagctgagcaatctgcagggcaccctagtgagcacctcttcatctgtgaggaatgtgggcgctgcaagtgcgtcccctgcacagcagctcgccctctcccctcctgctggctgtgcaaccagcgctgcctttgctctgctgagagcctcctcgattatggcacttgtctctgctgtgtcaagggcctcttctaccactgctccactgatgatgaagacaactgtgctgatgagccctgctcttgtgggcctagttcttgctttgtccgctgggcagccatgagcctcatctccctcttcctaccctgcctgtgctgctacctgcctacccgtggatgcctccatctgtgccagcagggctatgatagcctccggcgaccaggctgccgctgcaagaggcacaccaacactgtgtgcagaaagatctcttctggtagtgcacccttccccaaggcccaggaaaagtctgtatgaccttccaacaaggtggatccagagcttttctccttcgagtccccaacagcaaagcataggcctcatctttggagagggggaggagtgataaactagccaaagttagggcctctc

SEQ ID NO: 19 - DNA, 493 bp, human chromosome X genomic contig NT_025307.13 from Reference sequence project database (RefSeq)
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggaggaggagagagaggaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 20 - DNA, 493 bp, human chromosome Y genomic contig NT_079585.2 from Reference sequence project database (RefSeq); identical to SEQ ID NO: 19

SEQ ID NO: 21 - DNA, 476 bp, human chromosome X accesssion No. AADC01149041.1 from whole genome shotgun (WGS) database
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggagtaggagagagaggaggaggaggagaaagaaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttctgttttgttttgttttgtgttttctctgctgtgtcaaagaacaagacagaactatctc

SEQ ID NO: 22 - DNA, 439 bp, Chimp chromosome X Accession No. AADA01175381.1 from WGS database
ctgcttcggttcatctgtcagattggaggagagggagaccaaggtggaggcggaggcggaggcaaaagaggagggggaggaggtaaaggaggaagaaggggaggagggaaaggggagggcaagaggaggggaaggaaaatactggaggaggagggggaaaagaaacaggaggaggaggagaagggggaggagaaagaggcggaggaggaggagaaagaagaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 23 - DNA, 479 bp, Unmapped sequences Accession No. AADB01164924.1 from WGS database
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggaggagtaggagagagaggaggaggaggagaaagaaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttctgttttgttttgttttgtgttttctctgctgtgtcaaagaacaagacagaactatctc

SEQ ID NO: 24 - DNA, 479 bp, human chromosome Y Accession No. AADC01160617.1 from WGS database
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggaggagtaggagagagaggaggaggaggagaaagaaggaggaggtgaacaacttaccctgctgagctttctttgggaaatacgtccatcaagatttagatctgcctgtaaaatctatacaaagtatatgccactacaggtttgactcgccccctcccccgtttttttgttctgtcatgctctgttctgttgtgtcttgtgttttctctgctgtgtcaaagaacaagacagaactatctc

SEQ ID NO: 25 - DNA, 478 bp, human chromosome Y Accession No. AC025226.4 from High throughput genomic sequencing (HTGS) database
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggagtaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 26 - DNA, 475 bp, human chromosome 4 accesssion No. AC009620.4 from HTGS database
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagagagaggaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 27 - DNA, 479 bp, human chromosome X genomic contig NT_025307.13 (Fem#1 allele 1)
ctgcttcggttcatctgtcagtattttaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggagtaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 28 - DNA, 517 bp, human chromosome X genomic contig NT_025307.13 (Fem#1 allele2)
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggcggaggaggaggagaaagaagaggaggaggagaaagaggaggagtaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 29 - DNA, 459 bp, human chromosome X genomic contig NT_025307.13 (Fem#2 allele 1)
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaggaggagtaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgtttgttttgttttgtgttttctctgctgtgtcaaagaacaagacagaactatctc

SEQ ID NO: 30 - DNA, 511 bp, human chromosome X genomic contig NT_025307.13 (fem#2 allele 2)
  misc_feature (2)..(4): n is a, c, g, or t
  misc_feature (6)..(6): n is a, c, g, or t
  misc_feature (13)..(13): n is a, c, g, or t
  misc_feature (70)..(71): n is a, c, g, or t
cnnnancggttcntccctcagattggaggagagggagaccaaggtggaggcggaggcggaggcgaaagannagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggaggaggaggagagagaggaggaggaggagagagaggaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 31 - DNA, 488 bp, human chromosome X genomic contig NT_025307.13 (FEM#3 allele 1)
ctgcttcggttctctgtcattggagaaggagccaggtgggcggagcgaggcgaaaggagggggaggaggtaaaggagaaaggggaggagggaaaggggagggcaaagaggggaaggaatactggagggggagggggaagagaacaggaggaggaggagaagggggaggagaaagaggcggaggaggaggagaaagaggaggagtaggagaaagaggaggaggaggagagagaggaggaggaggagaaagagaaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgtttgttttgttttgtgttttctctgctgtgtcaaagaacaagacagaactatctc

SEQ ID NO: 32 - DNA, 477 bp, human chromosome X genomic contig NT_025307.13 (FEM#4 allele1)
ctgcttcggttcatctgtcagattggaggagagggagaccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaagaggaggaggagaaagaggagggtaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttatgttttgttttgtgttttctctgctgtgtcaaagaacaagacagaactatctc

SEQ ID NO: 33 - DNA, 474 bp, human chromosome X genomic contig NT_025307.13 (Fem#5 allele 1)
ctgcttcggttcatctgtcagattggaggagagggagccaaggtggaggcggaggcggaggcgaaagaggagggggaggaggtaaaggaggaagaaggggaggagggaaaggggagggcaagaggaggggaaggaaaatactggagggggagggggaagagaaacaggaggaggaggagaagggggaggagaaagaggcggaggaggaggagaaagaggaggagtaggagaaagaggaggaggaggagagagaggaggaggaggagaaagaggaggaggtgaacaacttaccctgctgagctttctttgggagatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 34 - DNA, 464 bp, human chromosome X genomic contig NT_025307.13 (Fem#5 allele2)
ctgcttcggttcatctgtcagattggaggagagggagccaaggtgagcgagcgagcgaaaaggagggggaggaggtaaaggaggaagaaggggaggagggaaaggggagggcaaaggaggggaaggaaaatctggagggggagggggaagagaaacaggaggaggaggagaagggggaggagaaaaggcggaggaggaggagaaagaggaggagtaggagaaagaggaggaggaggagagagaggaggaggaggagaaagaggaggaggtgaacaacttaccctgctgagctttctttgggaaatacgtccatcaagatttagatctgcctgtaaaatctatacaaagtatatgccactacaggtttgactcgccccctcccccgtttttttgttttgttttgttttgttttgttttgttttgtgttttctctgctgtgtcaaagaacaagacagaactatctc

SEQ ID NO: 35 - DNA, 24 bp, human chromosome X genomic contig NT_025307.13 (VBP1-XhoI)
ttcatgagaactacctcacctcct

SEQ ID NO: 36 - DNA, 27 bp, human chromosome X genomic contig NT_025307.13 (CLIC2-BsmAI)
tttgccatacagataaagaaaattaag

SEQ ID NO: 37 - DNA, 23 bp, human chromosome X genomic contig NT_025307.13 (SYBL1-XhoI)
gtcgaacgaacgtgaaacactca

SEQ ID NO: 38 - DNA, 21 bp, human chromosome X genomic contig NT_025307.13 (LH1)
tgaggcaggagaatcgggcag

SEQ ID NO: 39 - DNA, 20 bp, human chromosome X genomic contig NT_025307.13 (SYBL1-Rsa1)
atcaggttgcctggatttga

SEQ ID NO: 40 - DNA, 20 bp, human chromosome X genomic contig NT_025307.13 (IL9R-Sty1)
tgctgtgcacccagagatag

SEQ ID NO: 41 - DNA, 20 bp, human chromosome X genomic contig NT_025307.13 Str#11
agaaggatccaggtctgctg

SEQ ID NO: 42 - DNA, 13 bp, ARTIFICIAL SEQUENCE
  SYNTHESIZED
ggaggaggagaaa

SEQ ID NO: 43 - DNA, 15 bp, ARTIFICIAL SEQUENCE
  SYNTHESIZED
ggagaaagaagagga

SEQ ID NO: 44 - DNA, 17 bp, ARTIFICIAL SEQUENCE
  SYNTHESIZED
aggagaaagaggagggt

SEQ ID NO: 45 - DNA, 13 bp, ARTIFICIAL SEQUENCE
  SYNTHESIZED
gtaggaggagaga

SEQ ID NO: 46 - DNA, 25 bp, ARTIFICIAL SEQUENCE
  SYNTHESIZED
ggaggagagagaggaggaggaggag

SEQ ID NO: 47 - DNA, 25 bp, ARTIFICIAL SEQUENCE
  SYNTHESIZED
aggagagagaggaggaggaggagaa

SEQ ID NO: 48 - DNA, 17 bp, ARTIFICIAL SEQUENCE
  SYNTHESIZED
aggagagagaggaggag

SEQ ID NO: 49 - DNA, 25 bp, ARTIFICIAL SEQUENCE
  SYNTHESIZED
gagagagaggaggaggaggagaaag

SEQ ID NO: 50 - DNA, 17 bp, ARTIFICIAL SEQUENCE
  SYNTHESIZED
aggagaaagaggaggag

SEQ ID NO: 51 - DNA, 17 bp, ARTIFICIAL SEQUENCE
  SYNTHESIZED
aggaggtgaacaactta

SEQ ID NO: 52 - DNA, 15 bp, ARTIFICIAL SEQUENCE
  SYNTHESIZED
aggagtaggaggaga

SEQ ID NO: 53 - DNA, 13 bp, ARTIFICIAL SEQUENCE
  SYNTHESIZED
ggaggaggagaga

SEQ ID NO: 54 - DNA, 25 bp, ARTIFICIAL SEQUENCE
  SYNTHESIZED
aggagagagaggaggaggaggagag

SEQ ID NO: 55 - DNA, 25 bp, ARTIFICIAL SEQUENCE
  SYNTHESIZED
gagagagaggaggaggaggagagag

SEQ ID NO: 56 - DNA, 17 bp, ARTIFICIAL SEQUENCE
  SYNTHESIZED
aggagaaagaggcggag

SEQ ID NO: 57 - DNA, 21 bp, ARTIFICIAL SEQUENCE
  SYNTHESIZED
ctcctcctccgcctctttctc

SEQ ID NO: 58 - DNA, 15 bp, ARTIFICIAL SEQUENCE
  SYNTHESIZED
aggcggaggaggagg

SEQ ID NO: 59 - DNA, 15 bp, ARTIFICIAL SEQUENCE
  SYNTHESIZED
ggagaaagaaggagg

SEQ ID NO: 60 - DNA, 15 bp, ARTIFICIAL SEQUENCE
  SYNTHESIZED
aaagaaggaggaggt

SEQ ID NO: 61 - DNA, 11 bp, ARTIFICIAL SEQUENCE
  SYNTHESIZED
aaaaacggggg

SEQ ID NO: 62 - DNA, 15 bp, ARTIFICIAL SEQUENCE
  SYNTHESIZED
aacaaaaaaacgggg

SEQ ID NO: 63 - DNA, 17 bp, ARTIFICIAL SEQUENCE
  SYNTHESIZED
caaaacaaaaaaacggg

SEQ ID NO: 64 - DNA, 11 bp, ARTIFICIAL SEQUENCE
  SYNTHESIZED
aaaacaaaaaa

SEQ ID NO: 65 - DNA, 17 bp, ARTIFICIAL SEQUENCE
  SYNTHESIZED
caaaacaaaacaaaaaa

SEQ ID NO: 66 - DNA, 17 bp, ARTIFICIAL SEQUENCE
  SYNTHESIZED
caaaacaaaacaaaaca

SEQ ID NO: 67 - DNA, 19 bp, ARTIFICIAL SEQUENCE
  SYNTHESIZED
ttgttttgttttgttctgt

SEQ ID NO: 68 - DNA, 17 bp, ARTIFICIAL SEQUENCE
  SYNTHESIZED
caaaacaaaacagaaca

SEQ ID NO: 69 - DNA, 25 bp, ARTIFICIAL SEQUENCE
  SYNTHESIZED
aacagagcatgacagaacaaaaaaa

SEQ ID NO: 70 - DNA, 19 bp, ARTIFICIAL SEQUENCE
  SYNTHESIZED
ctgtcatgctctgttctgt

SEQ ID NO: 71 - DNA, 25 bp, ARTIFICIAL SEQUENCE
  SYNTHESIZED
gtaggagagagaggaggaggaggag

SEQ ID NO: 72 - DNA, 25 bp, ARTIFICIAL SEQUENCE
  SYNTHESIZED
gaaagagaaggaggaggagagagag

(19) United States
(12) Patent Application Publication
(10) Pub. No.: US 2007/0141577 A1
(43) Pub. Date: Jun. 21, 2007

(54) Method

(76) Inventor: Thomas F. Moore, Cork (IE)

Correspondence Address: JACOBSON HOLMAN;PROFESSIONAL LIMITED LIABILITY COMPANY, 400 SEVENTH STREET, N.W., WASHINGTON, DC, 20004, US

(21) Appl. No.: 10/938,515

(22) Filed: Sep. 13, 2004

Related U.S. Application Data
(60) Provisional application No. 60/501,863, filed on Sep. 11, 2003.

(30) Foreign Application Priority Data
Sep. 11, 2003 (IE) 2003/0661

(51) Int. Cl. C12Q 1/68

(52) U.S. Cl. 435/6

(57) ABSTRACT

A method of screening for genetic or epigenetic markers associated with autism or related disorders comprises the steps of providing a biological sample from a mammal; and testing the sample or genetic material isolated from the sample for genetic polymorphisms/mutations and/or epigenetic alterations. The polymorphism may be located in the Xq/Yq pseudoautosomal gene region and extends into the adjacent Xq28 gene region.

The invention relates to autism and related disorders.

Autism is a pervasive, behaviourally defined, developmental disorder consisting of a syndrome of delayed or abnormal speech development, impaired social interactions, and severely limited interests and activities. Autism is typically detected by 30 months of age, and is a life-long condition.

Structural brain abnormalities in autistics have been detected at postmortem, and by MRI scans in living subjects. While there is some evidence for increased brain size, or altered forebrain:hindbrain volume ratios, in autistic subjects, it is unclear how these changes relate to disease phenotype. There is also a strong association of autism with the genetically well-defined condition, tuberous sclerosis, however, this association is not correlated with the anatomic position of tubers in the brain. No clear evidence from tuberous sclerosis, therefore, consistently links disruption of a particular area of the brain to autism. However, Baron-Cohen et al. (2000) has proposed that the amygdala is one of several brain areas that is deregulated in autism.

Within the spectrum of autism-like disorders, there is considerable variation in the severity of symptoms or signs, such as mental retardation, which is present in 75% of autistic subjects. There may also be a variable presence or overlap with conditions defined as epilepsy, attention deficit/hyperactivity disorder (AD/HD), obsessive and compulsive behavioural disorders, neurofibromatosis, developmental coordination disorder, anxiety disorders, schizophrenia, bipolar disorder, depression, Asperger's syndrome, Rett syndrome, Fragile X, Turner's syndrome (XO karyotype), XYY syndrome and tuberous sclerosis (TS).

Outside of the core syndrome, as defined by the American Psychiatric Association in 1994, there are suggestive studies linking core autistic features to metabolic (Bolte, 1998), immune (Singh, 1996; Croonenberghs et al., 2002), and gastrointestinal disorders (Senior, 2002; Torrente et al., 2002).

There is strong evidence for a major genetic component in the causation of autism. This evidence includes twin studies, and the observed increased incidence of autistic features in the relatives of probands. Currently, a genetic model involving interactions between several susceptibility genes is favoured (Pickles et al, 2000). In support of this model, there are several genetic association studies linking particular alleles at several genetic loci to increased susceptibility to autism. However, such genetic associations tend to be weak, are frequently not replicated, and have little explanatory power in accounting for a key feature of autism and related disorders, the strongly male biased sex ratio among affected subjects. Pickles et al attributed the male-biased sex ratio to hormonal differences between males and females.

Interestingly, Baron-Cohen has proposed that the autistic spectrum represents an extreme form of the ‘male brain’ and links autism to altered digit-length ratios and prenatal exposure to testosterone (Manning et al., 2001).

A small number of genetic studies have specifically examined the sex chromosomes for the presence of autistic spectrum disorder susceptibility genes. Hallmayer et al. (1996) concluded that male-to-male transmission in extended pedigrees ruled out an exclusively X-linked mode of inheritance. Schutz et al. (2002) found no evidence of X-linkage using the affected sibling pair method. Jamain et al. (2002) examined the haplotype distribution of the non-recombining part of the Y chromosome in normal and autistic individuals but found no evidence of Y-linked susceptibility genes.

However, other studies have provided weak evidence of X chromosome linkage of autism susceptibility genes. In an association study, Petit et al. (1996) found linkage to X-linked marker DXS287 at Xq23. In a genomewide microsatellite scan of multiplex families Liu et al. (2001) found suggestive linkage at DXS470. Shao et al. (2002) also found evidence suggestive of X-linkage on the X chromosome. Jamain et al. (2003) identified mutations in the X-linked NLGN3 and NLGN4 genes in two families with autism.

Other attempts to determine the genetic basis of the autistic spectrum disorders have been ongoing and extensive, but largely unsuccessful using two established methods: 1). A candidate gene approach using genetic association studies, and 2) Genome wide scans (linkage analysis) in families.

Candidate gene approaches have low reproducibility and many candidates have been proposed and subsequently excluded following analysis in different populations or larger sample sizes. However, the imprinted Prader-Willi/Angelman region has been consistently associated with autism (Nurmi et al., 2001).

Linkage analysis has provided many candidate regions. A particularly interesting region is chromosome 7q31, which contains the language disorder gene FoxP2 (Newbury & Monaco, 2002; O'Brien et al., 2003).

Linkage analysis has also been carried our for attention deficit/hyperactivity disorder (ADHD) and Asperger's syndrome (AS). Some of the significant associations identified overlapped with loci previously implicated in autism (Bakker et al., 2003).

In ADHD a genetic susceptibility locus (SNAP-25), a member of the SNARE group of proteins, has been identified, which may explain some (but not a major component) of the susceptibility to this condition (Barr et al., 2000).

The available evidence for the autistic spectrum disorders can therefore be summarised to the effect that there are many candidate genetic loci identified in the literature for these strongly genetic disorders, but no strong causative genetic locus has been identified.

Autistic spectrum disorders (ASD) are costly in terms of care provision, and may be increasing in frequency. This view is controversial and may relate to wider syndrome definition and/or increased diagnosis. However, a recent study of Cambridgeshire school children aged 5-11 years found an incidence of 0.57% (Fiona et al., 2002). The Wakefield study (Wakefield et al., 1998) linking ASD to MMR vaccination has done immense damage to vaccination uptake. Therefore, apart from its inherent biological and medical importance, progress in defining the causes and mechanisms of ASD pathology is a pressing issue for wider aspects of public health.

A method of detecting the presence or susceptibility towards autism or related disorders would have major therapeutic and/or prophylactic potential.

### STATEMENTS OF INVENTION

According to the invention there is provided a method of screening for genetic or epigenetic markers associated with autism or related disorders comprising the steps of
isolating a biological sample from a mammal; and
testing the sample or genetic material isolated from the sample for genetic polymorphisms/mutations and/or epigenetic alterations.

Throughout the specification the term providing may be used instead of isolating.

A genetic marker is defined as a change in DNA sequence that is associated with a behavioural or other disorder. A genetic marker may also be understood as a mutation, a polymorphism, or a variant involving a change in DNA sequence associated with a behavioural or other disorder. An epigenetic marker is defined as a change in gene expression not involving a change in DNA sequence that is associated with a behavioural or other disorder. An epigenetic marker may comprise a change in chromatic structure or a covalent modification of DNA (such as cytosine methylation) that is associated with a behavioural or other disorder.

In one embodiment of the invention the polymorphism is located in the Xq/Yq pseudoautosomal gene region.

In another embodiment the polymorphism is located in the Xq/Yq pseudoautosomal gene region and extends into the adjacent Xq28 gene region.

In one embodiment the polymorphism is located in the Xq28 gene region adjacent to the Xq/Yq pseudoautosomal boundary.

The polymorphism may be a deletion of variable length.

Preferably the screening for deleted nucleic acids is carried out by a method selected from the group consisting of any one or more of enzymatic cleavage and southern hybridisation; in situ hybridisation using probes from the specified region; detection of loss-of-heterozygosity using genetic analysis of polymorphic RFLP and microsatellite markers; gene copy number analysis using real-time or other quantitative PCR technologies or DNA chip or array technologies.

In one embodiment of the invention the polymorphism involves a chromosomal translocation.

In another embodiment the polymorphism involves a chromosomal inversion.

In one embodiment the polymorphism involves a gene conversion event.

In one embodiment the polymorphism causes a reduction in gene dosage or gene expression, of some or all of the genes that map to the specified region.

In one embodiment of the invention the polymorphism causes an increase in gene dosage or gene expression, of some or all of the genes that map to the specified region.

In one embodiment of the invention the polymorphism causes an alteration in gene dosage, or in the temporal or spatial aspects of gene expression, of some or all of the genes that map to the specified region.

In one embodiment of the invention the polymorphism causes an alteration in gene dosage, or in the temporal or spatial aspects of gene expression, of the HSPRY3 gene.

In one embodiment of the invention the polymorphism causes an alteration in gene dosage, or in the temporal or spatial aspects of gene expression, of the SYBL1 gene.

In another embodiment the polymorphism involves a marker of epigenetic deregulation of gene expression. The marker of epigenetic deregulation of gene expression may be an alteration in patterns of DNA methylation or an alteration in patterns of nuclease sensitivity of DNA or chromatin.

In another embodiment the polymorphism involves a marker of epigenetic deregulation of gene expression comprising a change in the protein constitution of chromatin.

In one embodiment of the invention the marker of deregulation of gene expression is altered copy number or structure of DNA repeats in the HSPRY3 gene region.

In another embodiment of the invention the marker of deregulation of gene expression is alteration in the DNA sequence of the ‘MER31I c’ repeat in the HSPRY3 gene promoter.

In another embodiment of the invention the marker of deregulation of gene expression is alteration in the DNA sequence of the ‘GTTTT’ repeat downstream of the HSPRY3 gene transcriptional start site.

In another embodiment of the invention the marker of deregulation of gene expression is alteration of the DNA sequence downstream of the HSPRY3 gene protein coding region at the site of a recombination hotspot.

In another embodiment of the invention the marker of deregulation of gene expression is alteration of the DNA sequence downstream of the HSPRY3 gene protein coding region at the site of a transcript expressed in the amygdala or other regions of the brain.

In one embodiment of the invention the DNA sequence displaying abnormal levels of CpG methylation is the SYBL1 gene promoter-associated CpG island.

In one embodiment the marker of epigenetic deregulation of gene expression is loss-of-imprinting (reactivation) of the Y-linked copies of the HSPRY3, SYBL1 and TRPC6-like genes, alone or in combination.

In another embodiment the marker of epigenetic deregulation of gene expression is loss-of-imprinting (reactivation) of the Y-linked copy of the TRPC6-like gene.

The marker of epigenetic deregulation of gene expression may be increased or decreased mRNA or protein levels for the specified genes, in the absence of detectable DNA sequence polymorphisms.

In the method of the invention the biological sample may be selected from the group consisting of blood (including umbilical cord blood), saliva, semen, urine, amniotic fluid, placental biopsy, hair, tissue. The biological sample may be blood, a biopsy from a preimplantation stage embryo, a biopsy from the chorionic villus (extraembryonic tissue) of an implanted embryo (fetus) or fetal DNA or cells obtained from the serum of a pregnant mammal.

In one embodiment the mammal is a human.

In one aspect of the invention the biological sample is isolated from developmentally disabled children or the biological sample may be isolated from parents or relatives of developmentally disabled children.

The invention also provides a method for the treatment of autism and/or related disorders in children having genetic or epigenetic markers associated with autism or related disorders comprising the steps of:—
detecting in a biological sample genetic polymorphisms/mutations and/or epigenetic alterations; and
providing treatment in the form of any one or more of
   early behaviour training;
   early dietary interventions or manipulations; or
   pharmacological interventions.

The invention also provides a method for the treatment and/or prophylaxis of autism and/or related disorders in children having genetic markers associated with autism or related disorders comprising the steps of:—
detecting in a biological sample genetic polymorphisms/mutations and/or epigenetic alterations; and
providing any one or more of
   gene therapy;
   activation or reactivation of epigenetically silenced genes; or
   silencing or reducing gene expression at the mRNA or protein level.

In one embodiment of the invention the polymorphism is located in the Xq/Yq pseudoautosomal gene region and extends into the adjacent Xq28 gene region.

In another embodiment the polymorphism is located in the Xq28 gene region adjacent to the Xq/Yq pseudoautosomal boundary.

The invention also provides a method for the treatment and/or prophylaxis of autism and/or related disorders in children having genetic or epigenetic markers associated with autism or related disorders comprising activation or reactivation of epigenetically silenced genes in the Xq/Yq pseudoautosomal gene region.

The invention further provides a method for the treatment and/or prophylaxis of autism and/or related disorders in children having genetic or epigenetic markers associated with autism or related disorders comprising the step of silencing or reducing gene expression at the mRNA or protein level in the Xq/Yq pseudoautosomal gene region.

The invention also provides a method for selectively inhibiting or activating HSPRY3, AMD2; SYBL1, TRPC6-like, IL9R or CXYorf1 activity in a human host, comprising administering a compound which selectively inhibits or upregulates the activity of the gene products of HSPRY3, AMD2, SYBL1, TRPC6-like, IL9R or CXYorf1, to a human host in need of such treatment. The method may be used for the treatment and/or prophylaxis of autism and/or related disorders in children having genetic or epigenetic markers associated with autism or related disorders.

The invention provides a method for the treatment and/or prophylaxis of tetanus susceptibility, tuberous sclerosis (TS) or attention deficit hyperactivity disorder (ADHD) in patients having genetic or epigenetic markers associated with autism.

The invention also provides a method for the treatment and/or prophylaxis of tetanus susceptibility, tuberous sclerosis (TS) or attention deficit hyperactivity disorder (ADHD) in patients having genetic or epigenetic markers associated with autism or related disorders comprising activation or reactivation of epigenetically silenced genes in the Xq/Yq pseudoautosomal gene region.

The invention further provides a method for the treatment and/or prophylaxis of tetanus susceptibility, tuberous sclerosis (TS) or ADHD in patients having genetic or epigenetic markers associated with autism or related disorders comprising the step of silencing or reducing gene expression at the mRNA or protein level in the Xq/Yq pseudoautosomal gene region.

The invention also provides a method of screening for genetic or epigenetic markers associated with autism and related disorders comprising the steps of:
isolating a biological sample from a mammal;
isolating the Xq/Yq pseudoautosomal region (PAR) region in the sample; and
comparing the isolated Xq/Yq pseudoautosomal region (PAR) region with a control sequence, wherein a deletion, addition or mutation indicates a susceptibility to autism or related disorders.

The invention further provides a method for screening for genetic or epigenetic markers associated with autism and related disorders comprising the steps of:
isolating a biological sample from a mammal;
isolating the HSPRY3 gene promoter region in the sample; and
comparing the isolated HSPRY3 region with a control sequence, wherein a deletion, addition or mutation indicates a susceptibility to autism or related disorders.

Preferably the deletion, addition or mutation is an alteration in any one or more of the alleles listed in FIG. 3

Another aspect of the invention provides use of the Xq/Yq PAR and adjacent X-chromosome specific region comprising the entire DNA sequence listed in human chromosome X genomic contig NT_{—}025307.15.

Another aspect of the invention provides use of the Y chromosome region comprising the entire DNA sequence listed in human chromosome Y contig NT_{—}079585.2.

Another aspect of the invention provides use of the Y chromosome region comprising the entire DNA sequence listed in human chromosome Y WGS clone AADC01160617.1.

One aspect of the invention provides use of the Xq/Yq PAR and adjacent X-chromosome specific region comprising the entire DNA sequence listed in human chromosome X genomic contig NT_{—}025307.13 in the detection of autism or autism related disorders in patients.

The invention further provides a DNA sequence comprising a nucleic acid sequence selected from any one or more of SEQ ID Nos. 1 to 13 or SEQ ID Nos. 35 to 41.

The invention further provides a DNA sequence comprising a nucleic acid sequence selected from any one or more of Seq ID Nos. 14 to 18 or Seq ID Nos. 27 to 34.

One aspect of the invention provides use of LH1 simple tandem repeat as a genetic marker associated with autism or autism related disorders.

A further aspect of the invention provides use of XhoI, BsmAI, SYBLI-XhoI, RsaI, StyI or HinfI RFLPs as genetic markers associated with autism or related disorders.

Another aspect of the invention provides use of polymorphisms of the ‘MER31I c’ repeat in the promoter region of the HSPRY3 gene as genetic markers associated with autism or related disorders.

Another aspect of the invention provides use of the polymorphic A/G diallelic marker in the HSPRY3 gene coding region as a genetic marker associated with autism or related disorders.

A further embodiment of the invention provides use of polymorphisms of the DNA or RNA sequences or encoded protein sequences of transcription factors (transcriptional enhancers or repressors) or chromatin proteins that bind to regulatory regions of genes in the Xq/Yq PAR and adjacent X-chromosome region.

A further embodiment of the invention provides use of polymorphisms of regulatory RNA sequences (including microRNAs) that bind to the regulatory regions of genes in the Xq/Yq PAR and adjacent X-chromosome region.

A further embodiment of the invention provides use of polymorphisms of DNA, RNA or protein sequences associated with factors that interact with the regulatory regions of the SYBL1 or HSPRY3 genes.

A further embodiment of the invention provides use of polymorphisms of DNA, RNA or protein sequences associated with factors that interact with the ‘MER31I c’and ‘GTTTT’ repeat regions of the HSPRY3 gene.

Another aspect of the invention provides use as genetic markers associated with autism or related disorders of alterative polymorphisms of the MAZ/PUR1 gene DNA or protein sequence.

Another aspect of the invention provides use as genetic markers associated with autism or related disorders of alterative polymorphisms of the sex determining region Y (SRY) gene DNA or protein sequence.

Another aspect of the invention provides use as genetic markers associated with autism or related disorders of alterative polymorphisms of the progesterone receptor gene DNA or protein sequence.

Another aspect of the invention provides use as genetic markers associated with autism or related disorders of alterative polymorphisms of the vitamin D receptor gene DNA or protein sequence.

Another aspect of the invention provides use as genetic markers associated with autism or related disorders of alterative polymorphisms of the Retinoid X receptor gene DNA or protein sequence.

Another aspect of the invention provides use as genetic markers associated with autism or related disorders of alterative polymorphisms of the Fkh-domain factor FKHRL1 (FOXO) gene DNA or protein sequence.

Another aspect of the invention provides use as genetic markers associated with autism or related disorders of alterative polymorphisms of the Nerve growth factor-induced protein C gene DNA or protein sequence.

Another aspect of the invention provides use as genetic markers associated with autism or related disorders of alterative polymorphisms of GAGA-Box binding factor genes DNA or protein sequence.

Another aspect of the invention provides use as genetic markers associated with autism or related disorders of alterative polymorphisms of the Gut-enriched Krueppel-like factor gene DNA or protein sequence.

Another aspect of the invention provides use as genetic markers associated with autism or related disorders of alterative polymorphisms of the Barbiturate-inducible element gene DNA or protein sequence.

Another aspect of the invention provides use as genetic markers associated with autism or related disorders of alterative polymorphisms of the v-MYB, variant of AMV v-myb gene DNA or protein sequence.

Another aspect of the invention provides use as genetic markers associated with autism or related disorders of alterative polymorphisms of the Multifunctional c-Abl src type tyrosine kinase gene DNA or protein sequence.

Another aspect of the invention provides use as genetic markers associated with autism or related disorders of alterative polymorphisms of the Glucocorticoid receptor C2C2 zinc finger protein gene DNA or protein sequence.

Another aspect of the invention provides use as genetic markers associated with autism or related disorders of alterative polymorphisms of the ‘TCF11/MafG heterodimers, binding to subclass of AP1 sites’ gene DNA or protein sequence.

Another aspect of the invention provides a method of assessing the personality of a patient or their susceptibility to autism or related disorders comprising the step of genotyping the ASD locus comprising genes in the Xq/Yq PAR region.

The method of the invention may be used in early behaviour training, early dietary interventions or manipulations, pharmacological interventions, gene therapy, activation or reactivation of epigenetically silenced genes or silencing or reducing gene expression at the mRNA or protein level in children who have genetic or epigenetic markers associated with autism or related disorders.

Samples may be isolated from children believed to have autism or related disorders or from clinically normal children. The biological sample may also be provided or isolated from parents or relatives of clinically normal children who have genetic markers associated with autism or related disorders.

### BRIEF DESCRIPTION OF THE DRAWINGS

The invention will be more clearly understood from the following description thereof, given by way of example only, in which:—

FIG. 1 is a schematic representation of the Xq/Yq pseudoautosomal region (PAR) which exhibits an unusual form of genetic/epigenetic regulation. The full sequence listing can be obtained from the Human Genome Sequencing Project, available on the NCBI website at (http://www.ncbi.nlm.nih.gov/entrez/query.fcgi?db=nucleotide&cmd=search &term=NT_{—}025307.13).

The PAR consists of approximately 300 Kb and contains the HSPRY3, AMD2 (S-AdometDC-like), SYBL1, TRPC6-like, IL9R and CXYorf1 genes (AMD2 may be a non-expressed pseudogene). X chromosome-specific genes adjacent to the PAR include the TMLHE, CLIC2, RAB39B and VBP1 genes. HSPRY3 and SYBL1 undergo random X-inactivation in females, but preferential Y-inactivation in males;

The entire region is ˜0.8 Mb and based on contig NT_{—}025307.13 (X-chromosome). The gene size scale is an approximation. Horizontal arrows indicate direction of transcription. CXYorf1 function unknown but found near several telomeres. AMD2 is a pseudogene—not known if expressed. Vertical arrows indicate positions of polymorphic small tandem repeats (STRs) and restriction fragment length polymorphisms (RFLPs; restriction enzymes in italics). ‘MER31I c’ is a DNA repeat upstream of the HSPRY3 transcriptional start site. HSPRY3-SNP is an A/G diallelic marker in the HSPRY3 coding region. NT_{—}025307.15 is an updated version of NT_{—}025307.13 (released August 2004). There are more genes included in the X-specific region as follows: hepatitis C virus core-binding protein 6, mature T-cell proliferation 1, c6.1A, LOC401622, H2AFB. Also, the orientation of the TMLHE gene has been reversed.

FIG. 2 is a table listing the Coriell autism family collection and the genotype of each individual at genetic loci in the Xq/Yq PAR and adjacent X chromosome-specific region;

FIG. 3 is a table showing the PCR primer sequences spanning the polymorphic sites of restriction enzyme fragment length polymorphisms (RFLP) and simple tandem repeats (STRs) identified. All sequences are derived from genomic contig NT_{—}025307.13, or from sources listed under the reference column including Matarazzo et al 2002 & Li and Hamer 1995; FIG. 4 is a table showing genotype frequencies at polymorphic sites in the Xq/Yq PAR and adjacent X chromosome-specific region in subsets of autistic and control groups;

FIGS. 5, 6 and 7 are tables showing the results of statistical analysis of genotype frequencies for selected polymorphic genetic loci in the Xq/Yq PAR. Specifically, the ‘within group’ distribution of homozygotes and heterozygotes is compared between various affected and control (unaffected) population groups. The analysis shows that there is a statistically significant difference in the distribution of homozygotes and heterozygotes in the affected, compared to the control (unaffected) groups for markers in the SYBL1 gene region. These results indicate a loss-of-heterozygosity (LOH) for the four SYBL1 associated markers: SYBL1 STR#1B, SYBL1 STR#2B, LH1, SYBL1-RsaI. The flanking markers SYBL1-XhoI and IL9R-StyI are unaffected.

FIG. 8 is a multiple alignment of DNA sequences from the promoter region of the HSPRY3 gene spanning the ‘MER31I c’ and ‘GTTTT’ repeats. The sequences are derived from the public databases and from our single pass sequencing of cloned PCR products from genomic DNA of normal Irish women. The sequences establish that the major polymorphisms in the region occur in the ‘MER31I c’ and ‘GTTTT’ repeats.

FIG. 9 shows the evidence for a putative recombination hotspot at the 3′ end of the HSPRY3 coding sequence region (CDS) and the 5′ end of the HSPRY3 3′ untranslated region (UTR). The HSPRY3-SNP (P) and HSPRY3-HinfI (Q) SNPs are separated by 156 bp within a PCR product (FIG. 3). PCR and single-pass sequencing was performed on both parents and an autistic individual from thirteen families from the Coriell Autism Resource. The linked alleles on each of the two sex chromosomes are displayed in the format P-Q/P-Q. The data suggest that there is a recombination hotspot between the two markers because all four recombination products (A-T, G-G, A-G, G-T) are observed.

FIG. 10 shows the HSPRY3 promoter region genotypes of normal females of Irish origin, normal young males from the Coriell Ageing Resource, and members of seven families from the Coriell Autism Resource. The PCR primers used are listed in FIG. 3 under ‘MER31I c; and

FIG. 11 shows PCR products obtained using primers listed in FIG. 3 under ‘MER31I c’ from Coriell Autism Resource family run on an agarose gel. PCR analysis using primers (FIG. 3) spanning ‘MER31I c’ and ‘GTTTT’ repeats of HSPRY3 promoter. Samples: genomic DNA from family comprising father (1), mother (2), male proband (3), affected male sib (4) from Coriell Autism Resource. Arrowheads indicate PCR products for clarity. Arrow indicates novel PCR product in affected males, which is not found in parents.

### DEFINITIONS

A genetic alteration is taken to include polymorphisms or mutations and/or epigenetic alterations.

The term polymorphism is intended to include all possible alterative variants of a DNA, RNA or protein sequence. It is analogous to the term ‘mutation’ and is often, but not exclusively, used to refer to a variant sequence that is present at a frequency of greater than 1% in the population.

A mutation is taken to include deletions, additions or insertion or substitutions of one or more of the nucleotide or amino acid residues.

A deletion refers to a change in either nucleotide or amino acid sequence and results in the absence of one or more nucleotides or amino acid residues. An insertion or addition refers to a change in a nucleotide or amino acid sequences that results in the addition of one or more nucleotide or amino acid residues as compared with the naturally occurring molecule. A substitution refers to the replacement of one or more nucleotides or amino acids by different nucleotides or amino acids.

Loss-of-imprinting or reactivation is taken to include the pathological, experimental or therapeutic induction of gene expression at a genetic locus that was previously silenced (transcriptionally inactive) due to epigenetic modifications of DNA or chromatin.

A diallelic marker is taken to include a single nucleotide polymorphism where there are two variants present.

Transcription factors are taken to include transcriptional enhancers or repressors.

An allele or allelic sequences is an alternative form of a nucleic acid sequence. Alleles may result from at least one mutation in the nucleic acid sequences and may yield altered mRNAs or polypeptides whose structure of function may or may not be altered. Common mutational changes which give rise to alleles are generally ascribed to natural deletions, additions or substitutions of nucleotides.

### DETAILED DESCRIPTION

We have identified a major ASD locus comprising genes in the Xq/Yq pseudoautosomal region (PAR). In principle, deregulation of genes at this locus provides an explanation for the phenotypic variability of the autistic spectrum, the male-biased sex ratio of affects, and also provides plausible mutational mechanisms.

The locus comprising a number of genes provides an answer to the diverse disturbances and reasons for the failure of standard genetic mapping studies to locate such a locus. Deregulation of genes located in the Xq/Yq PAR and adjacent X chromosome-specific (Xq28) region may provide an explanation for many of the features of ASD.

The region can account for male-biased affected sex ratios due to its unusual genetic/epigenetic regulation. Deregulation of the genes in the region might be involved in, for example: structural brain abnormalities (HSPRY3, SYBL1); abnormal neuron function (CLIC2, SYBL1, TRPC6-like); metabolic/mitochondrial disturbances (TMLHE); immune dysfunction (IL9R); or other gene deregulation through effects on chromatin structure (TMLHE).

The method of the invention provides for screening of subjects for genetic polymorphisms and epigenetic markers associated with autism and related disorders. The method involves isolating DNA from a mammal, specifically a human, and testing the sample for (i) deletions and other structural genomic rearrangements in the Xq/Yq pseudoautosomal gene region, and in the adjacent Xq28 gene region; (ii) polymorphic DNA markers in the Xq/Yq pseudoautosomal gene region, and in the adjacent Xq28 gene region, associated with autism and related disorders; (iii) alterations in the epigenetic regulation (including DNA methylation) of genes in the Xq/Yq pseudoautosomal gene region, and adjacent Xq28 gene region; and/or (iv) absence or downregulation, and over-expression (i.e. upregulation) of genes in the Xq/Yq pseudoautosomal gene region, and adjacent Xq28 gene region; altered temporal or spatial patterns of regulation or expression of genes in the Xq/Yq pseudoautosomal gene region, and adjacent Xq28 gene region.

The presence of such alterations indicates that the subject is afflicted with autism or related disorders, is at greater risk of developing autism or related disorders or is at greater risk of transmitting autism or related disorders to progeny.

DNA sequences (isolated DNA) have been characterised comprising restriction enzyme fragment length polymorphisms (RFLPs), polymorphic microsatellite (dinucleotide repeat) sequences, and other repeat sequences such as the ‘MER31I c’and ‘GTTTT’ repeats in the HSPRY3 gene useful for genetic mapping in the Xq/Yq pseudoautosomal gene region and in the adjacent Xq28 gene region. The sequences are publicly available on the Human Genome Sequence database. Regions were selected that are known to be polymorphic (known RFLPs, known simple tandem repeats (STRs)), or STRs were selected which were not known to be polymorphic and primers were designed spanning them (FIG. 3).

The allelic structure of STRs 3, 5, 9 & 10; SYBL1-STR#1B, SYBL1-STR#2B; ‘MER31I c’ repeat; and HSPRY3-SNP was determined. They are polymorphic and the alleles occurring are given in FIG. 3.

We also found in the present invention that the LH1 STR marker as well as XhoI, BsmAI, SYBL1-XhoI, RsaI, StyI, and HSPRY3-HinfI RFLPs may be used to study autism.

The identities of genes previously mapped to the Xq/Yq pseudoautosomal gene region and the adjacent Xq28 gene region, the deregulation of which is thought to explain the observed biochemical, clinical, and genetic (particularly male-biased sex ratio) features of autism and related disorders: CXYorf1, IL9R, TRPC6-like, SYBL1, AMD2, HSPRY3, TMLHE, CLIC2, RAB39B, VBP1 are also described.

The identification of the ASD locus provides valuable methods of developing therapeutic strategies for autism and related disorders.

The finding that a specific genetic locus may be implicated in the majority of ASD causation has important application in a number of areas such as for example (i) diagnostic and prognostic tests; (ii) scope for further study in relation to pathogenesis and therapeutics; or (iii) a platform for providing reassurance in relation to public health issues such as vaccination.

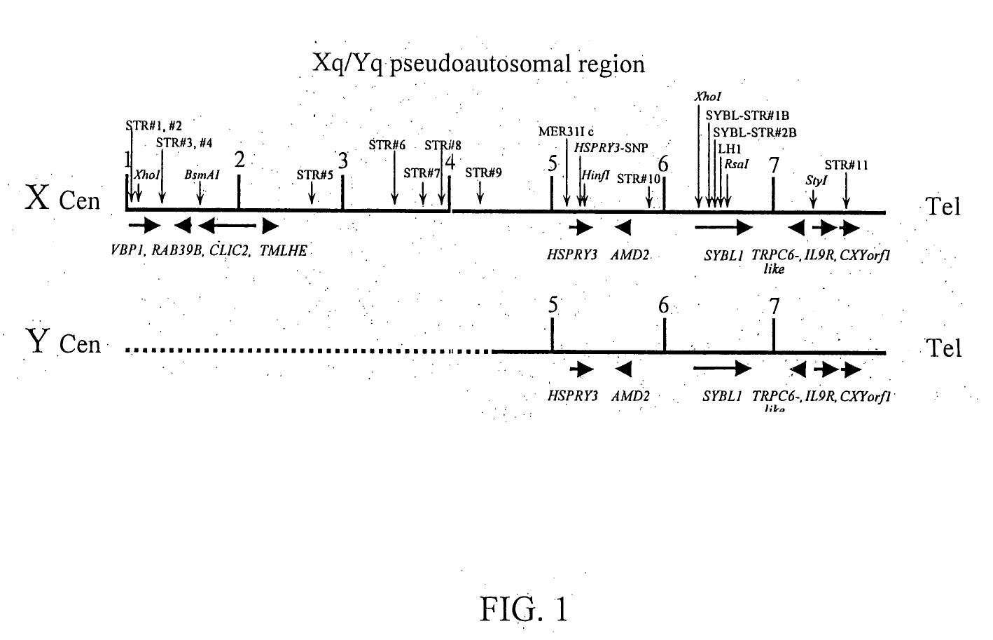

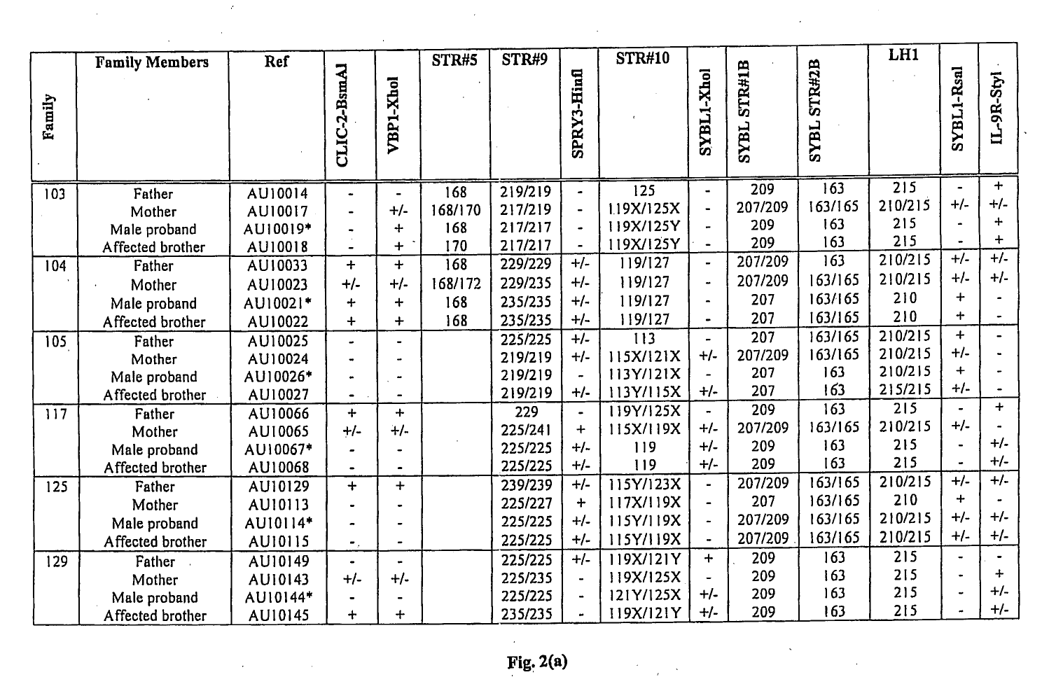

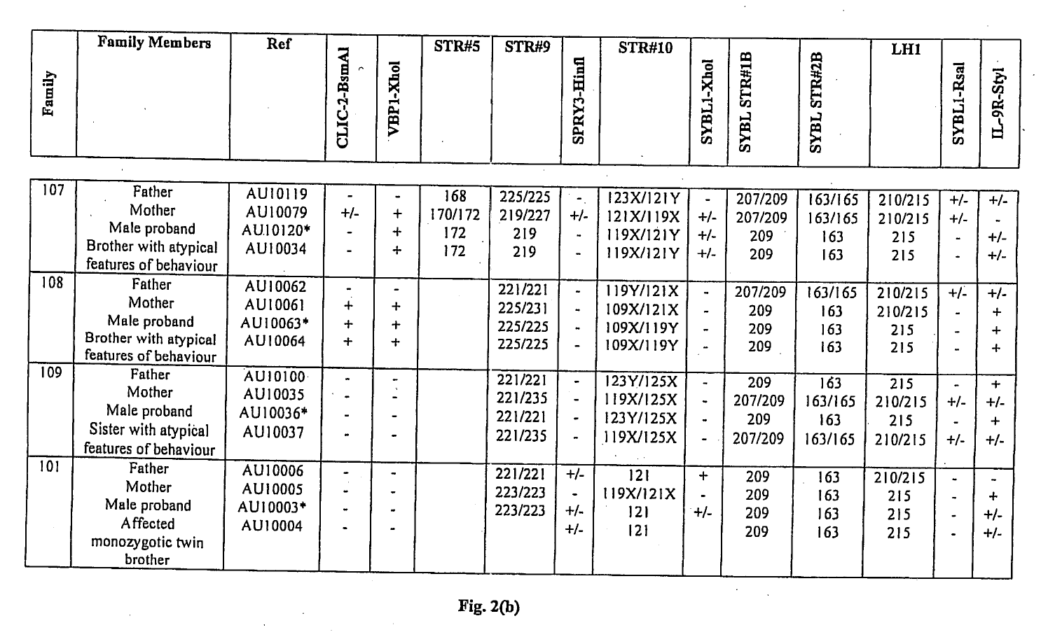

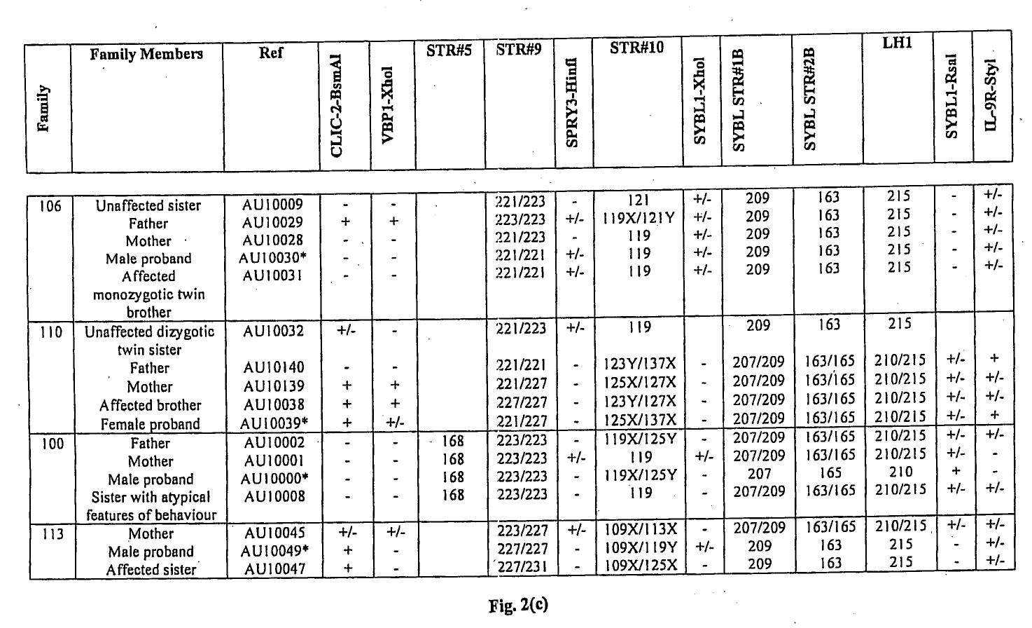

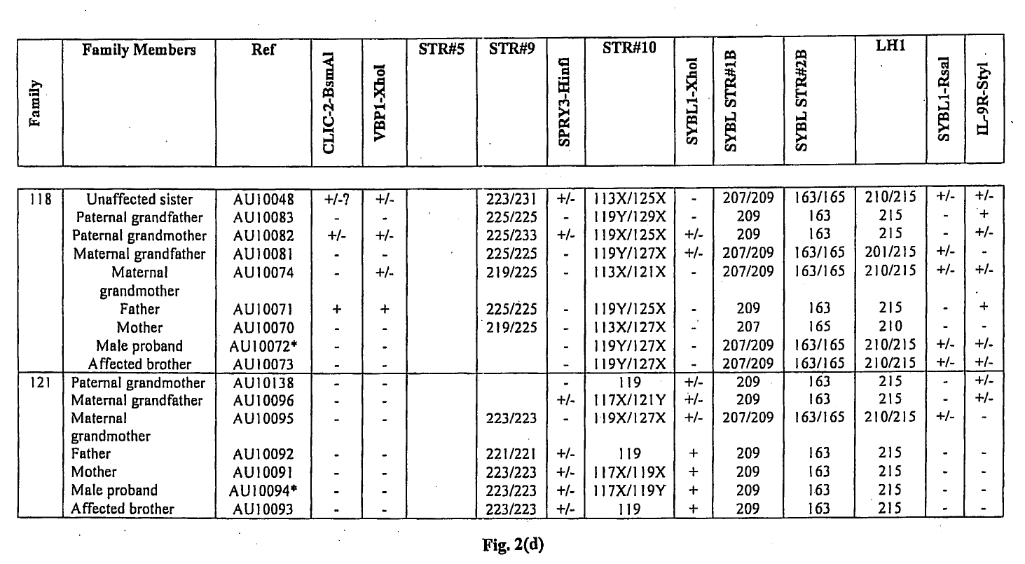

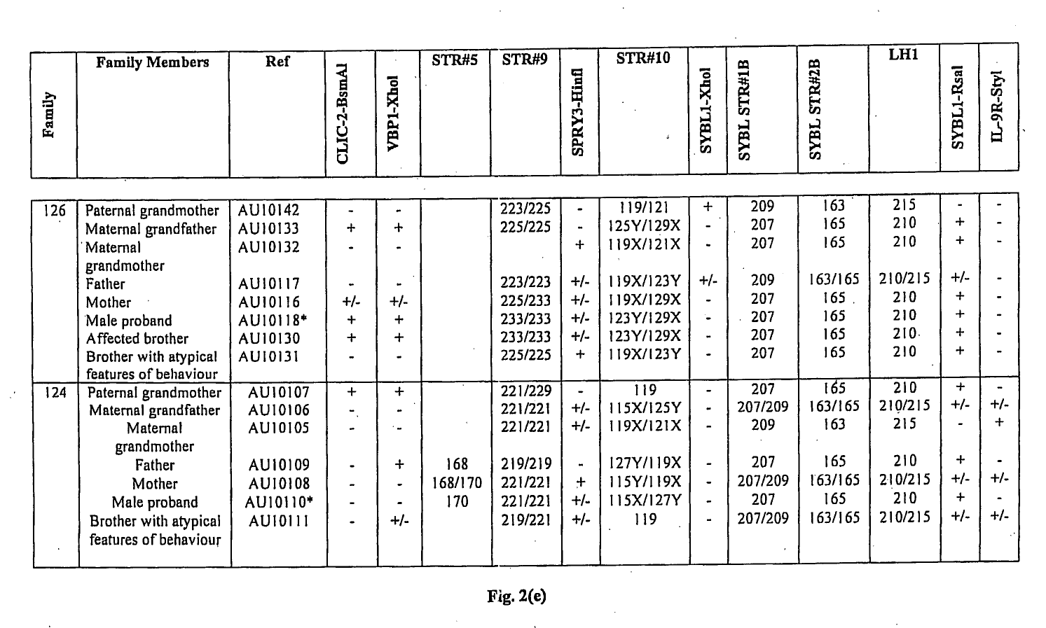

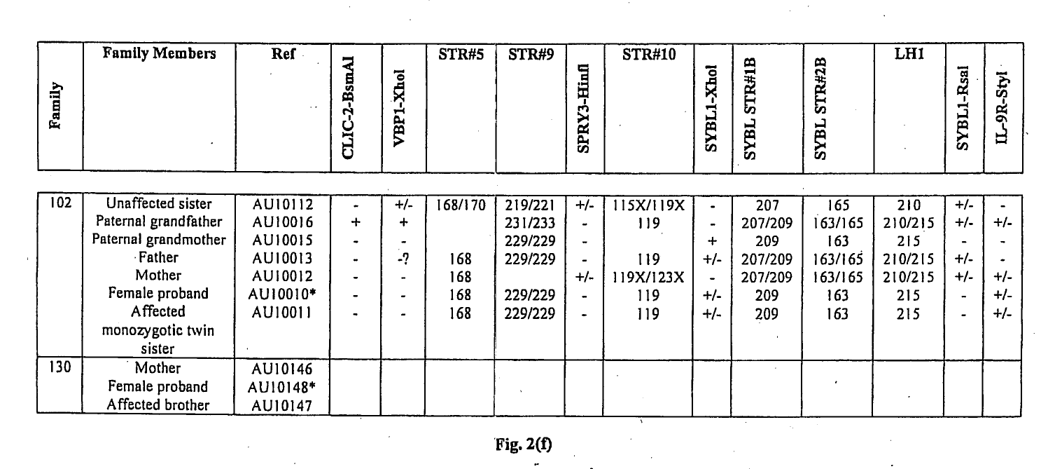

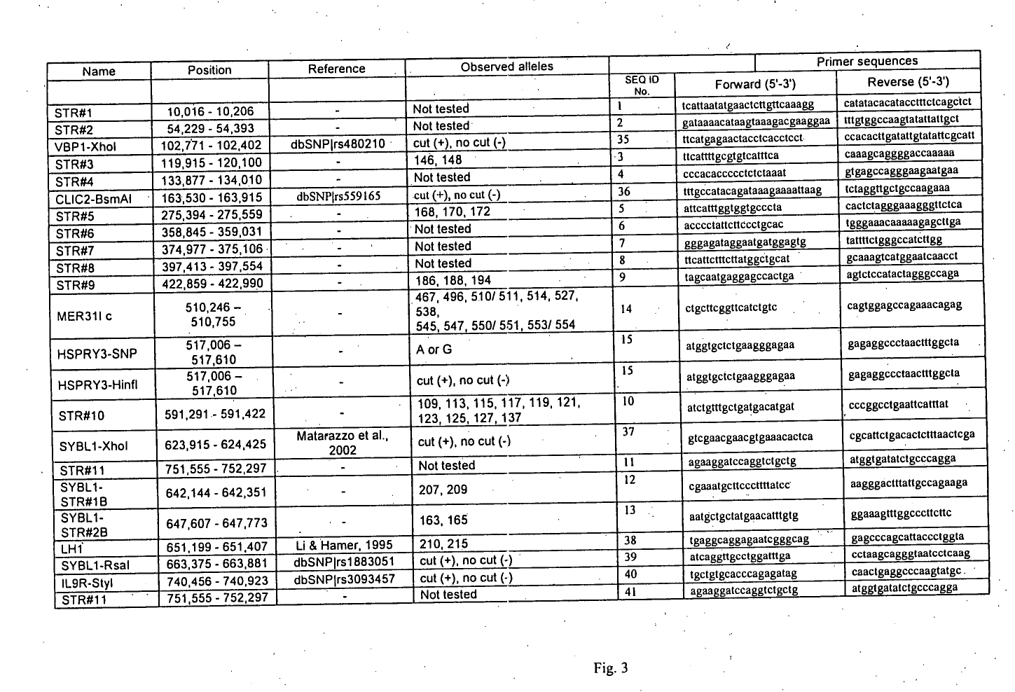

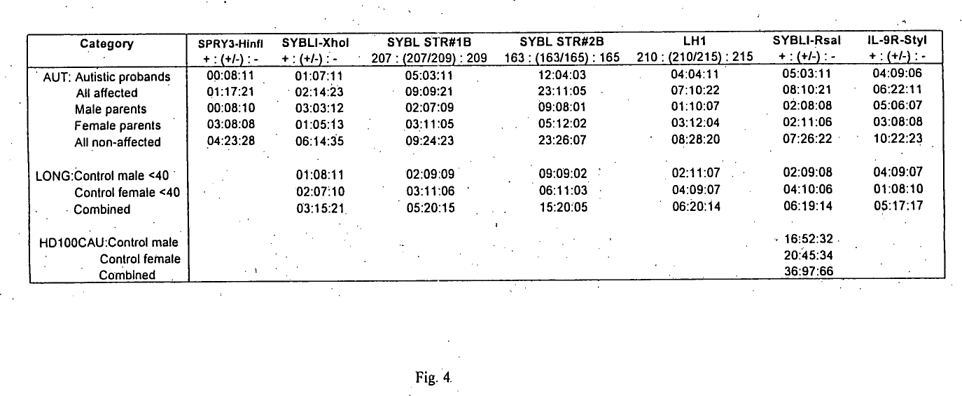

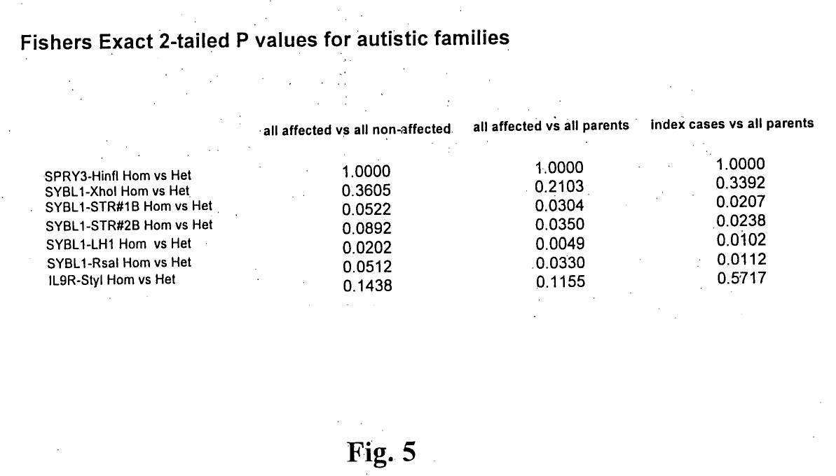

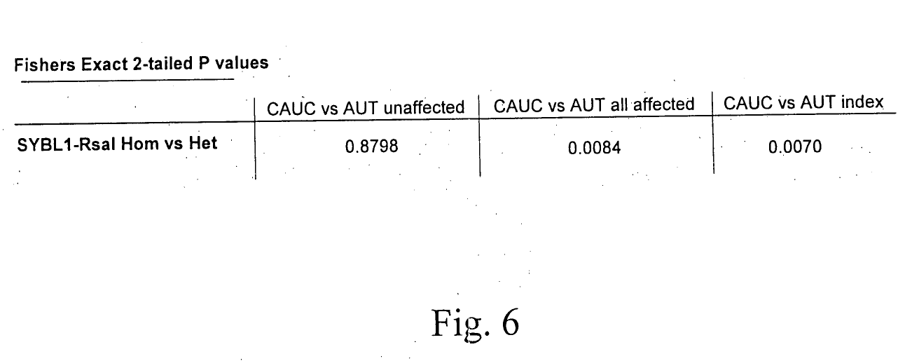

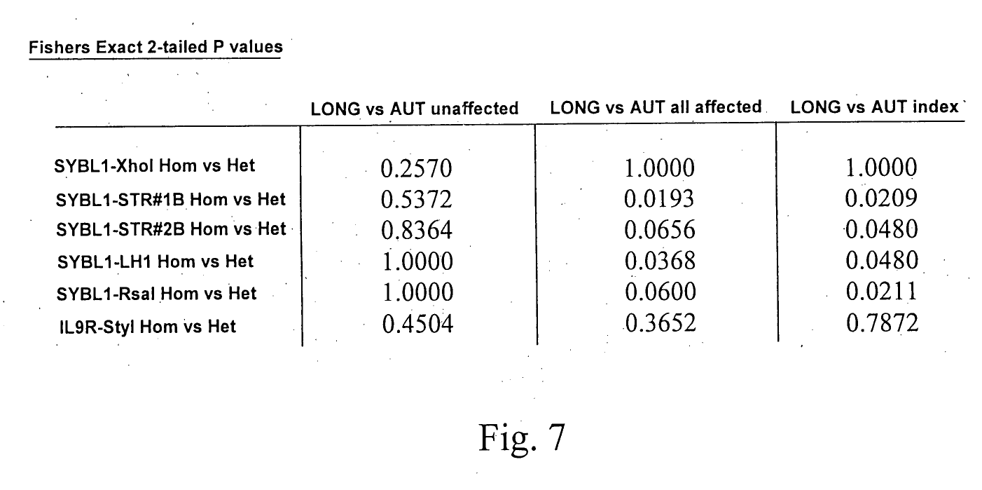

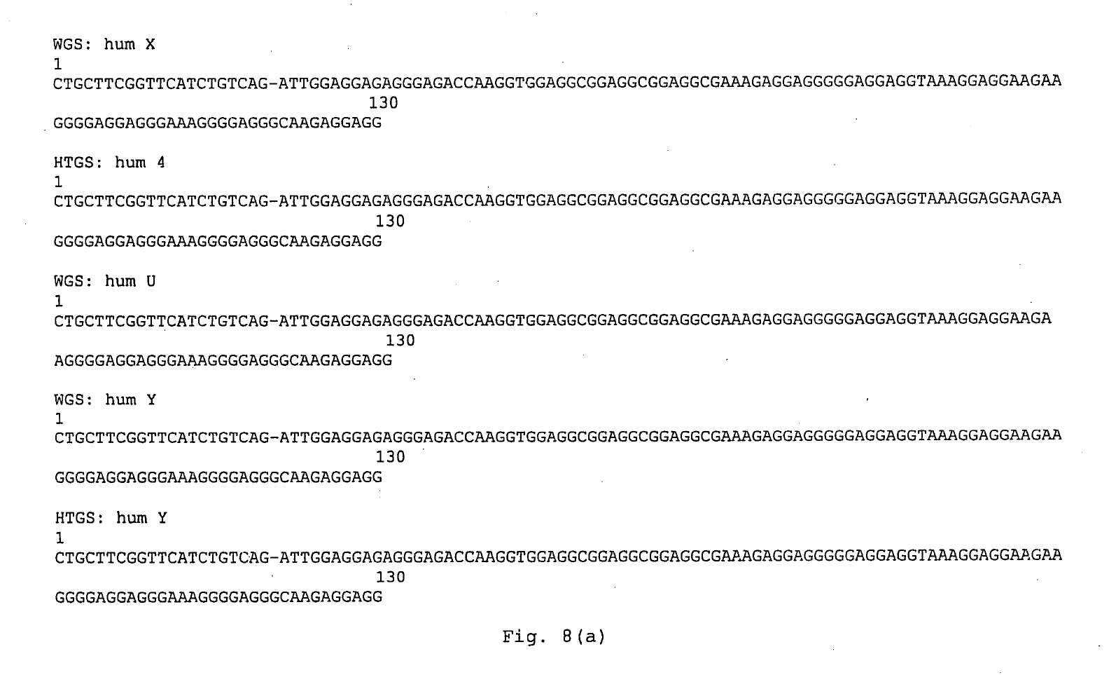

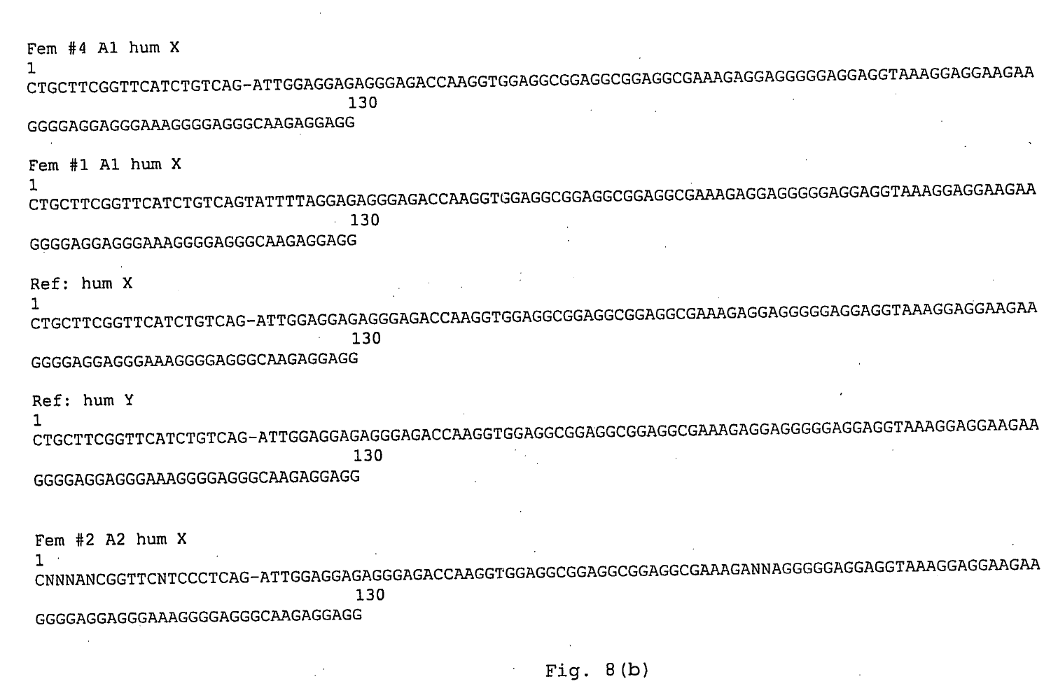

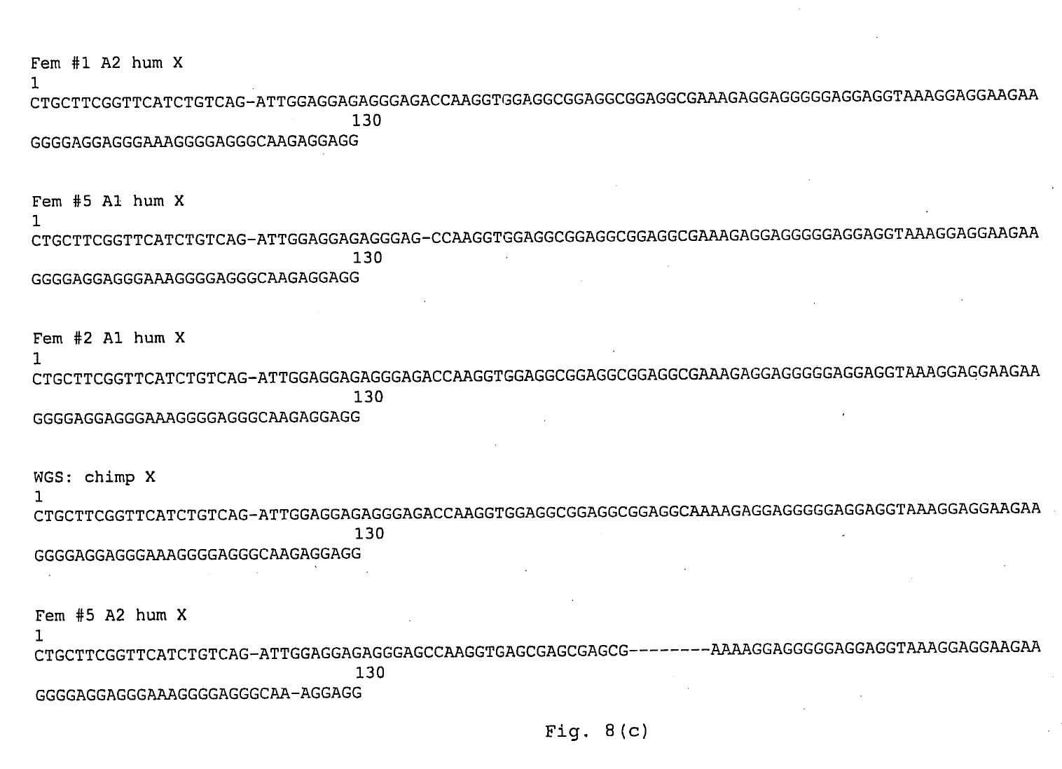

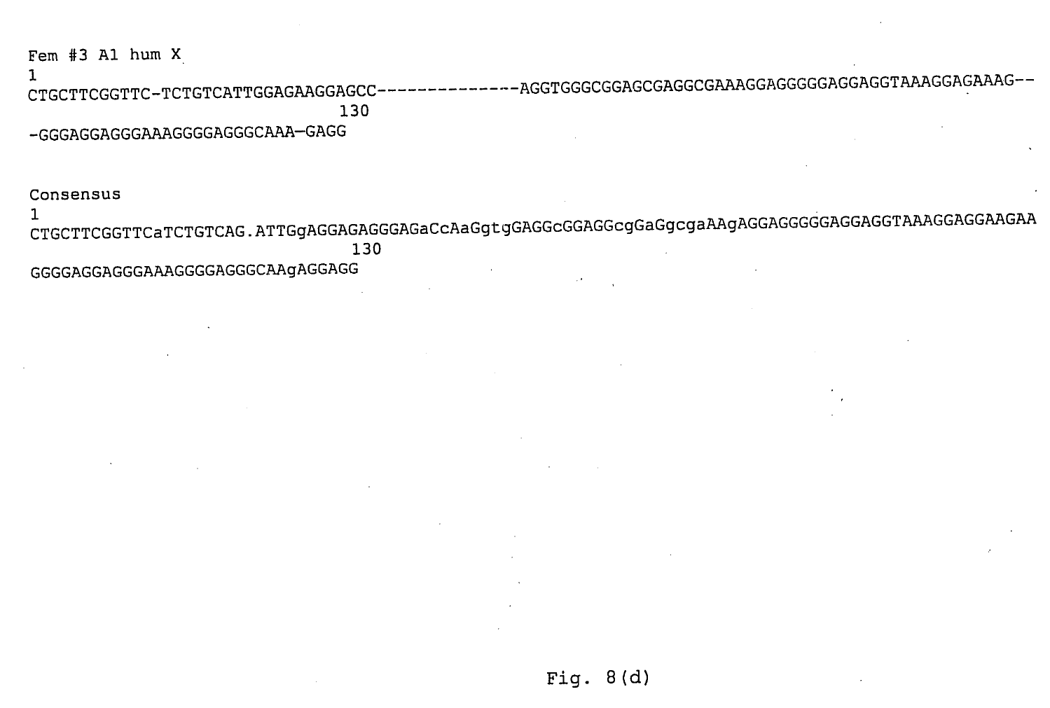

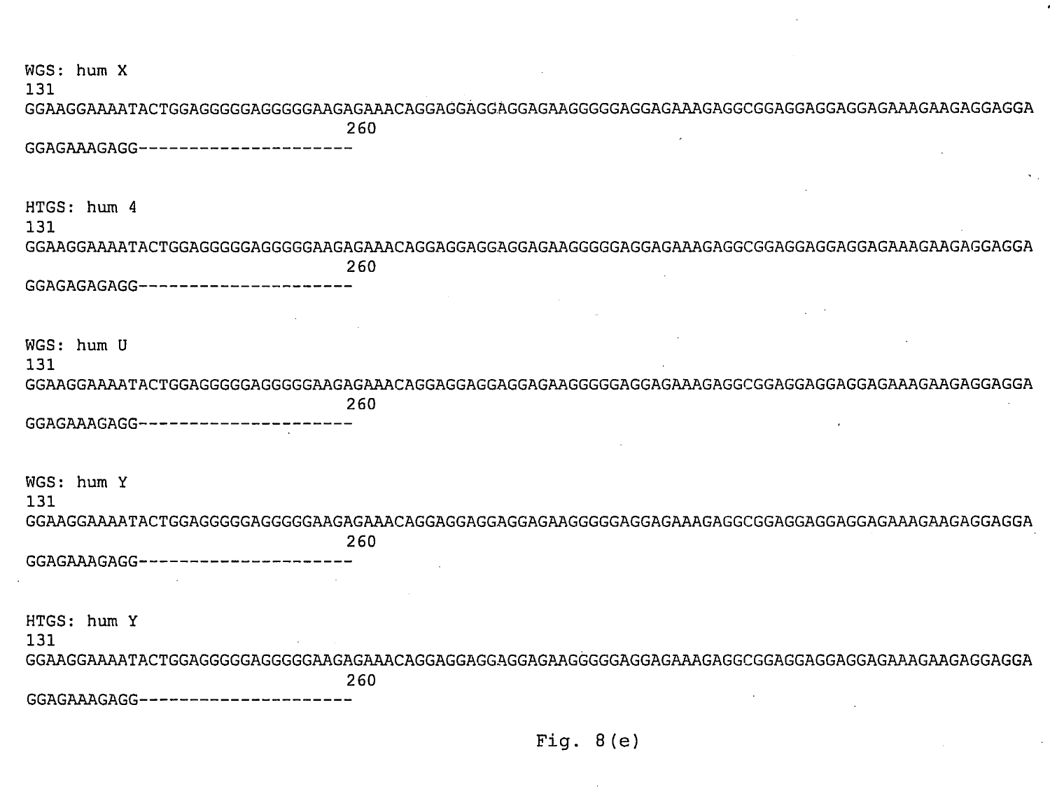

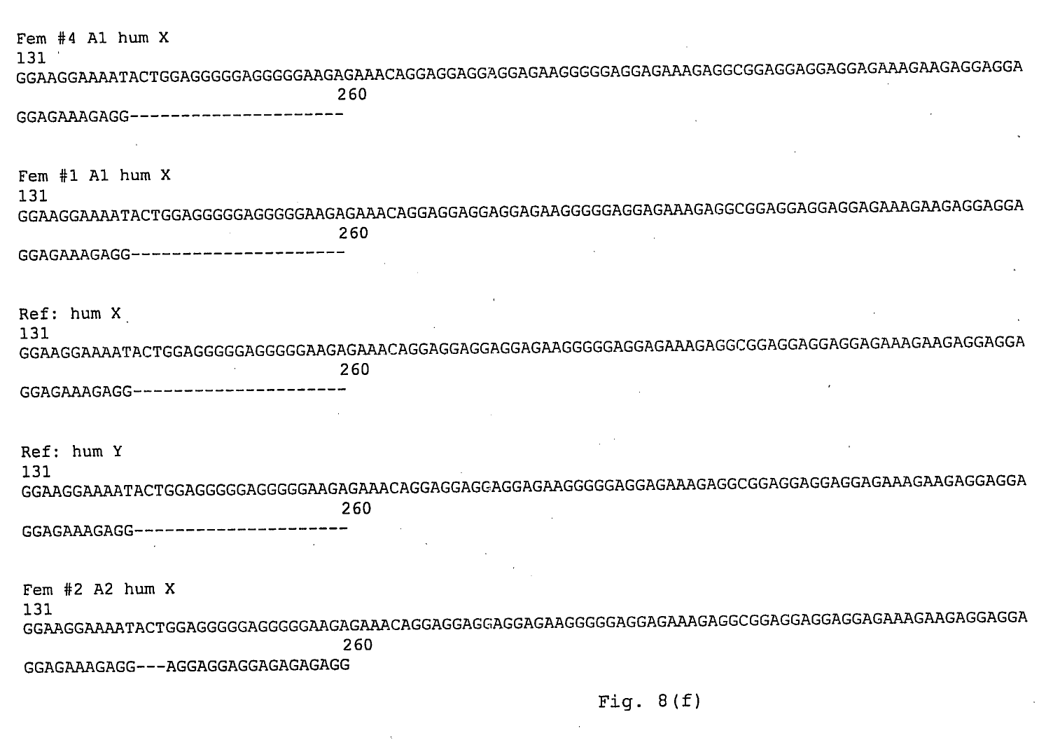

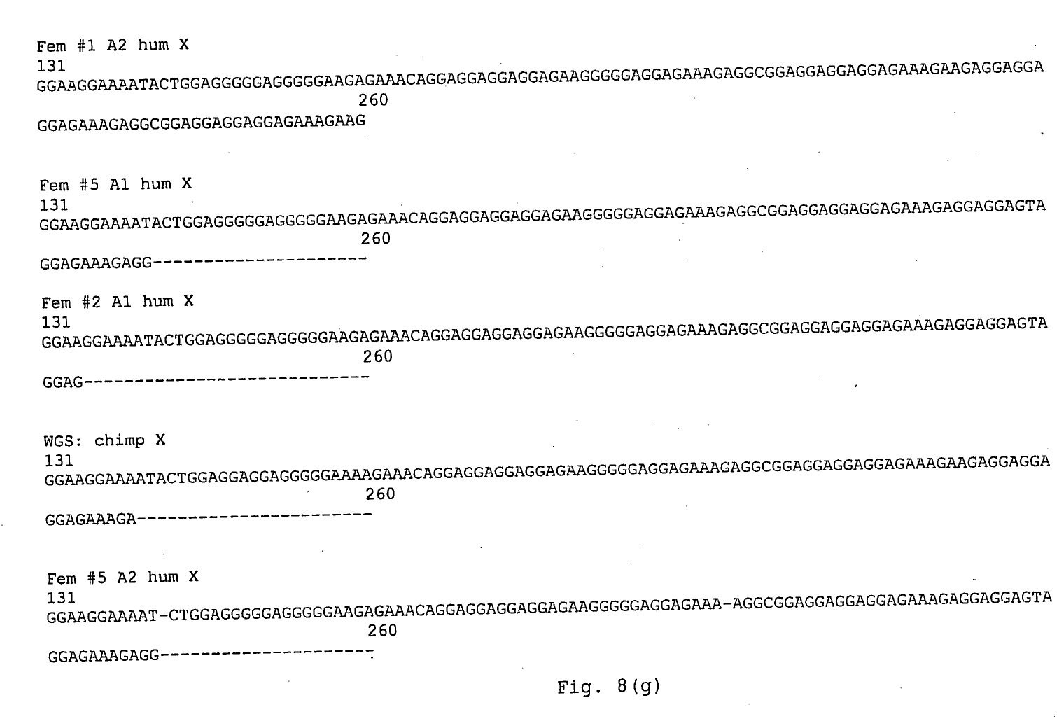

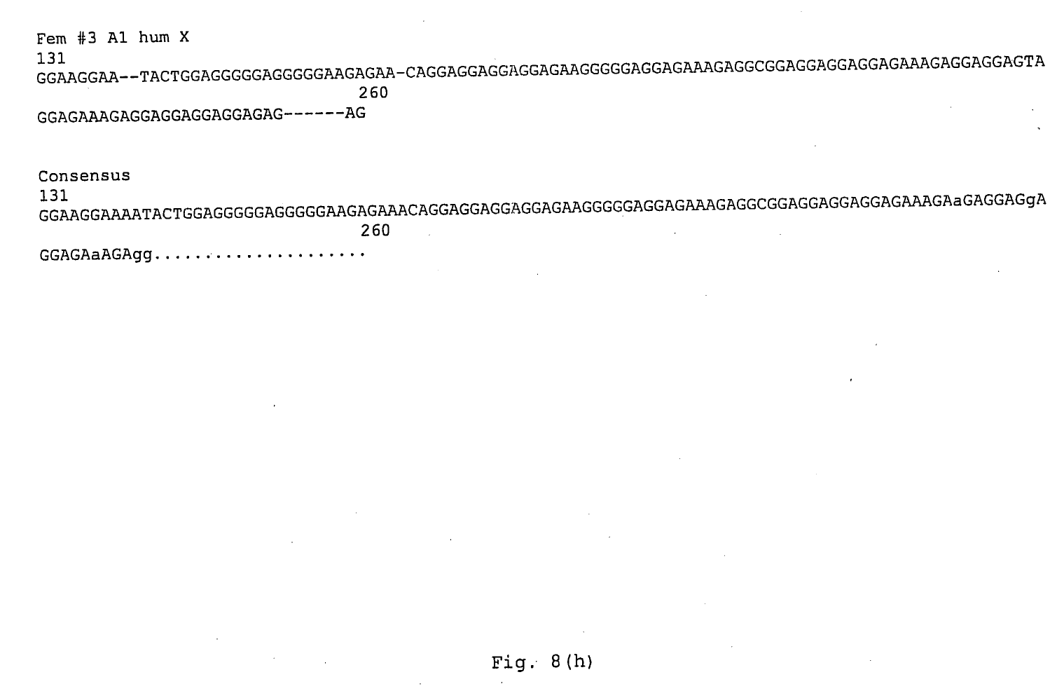

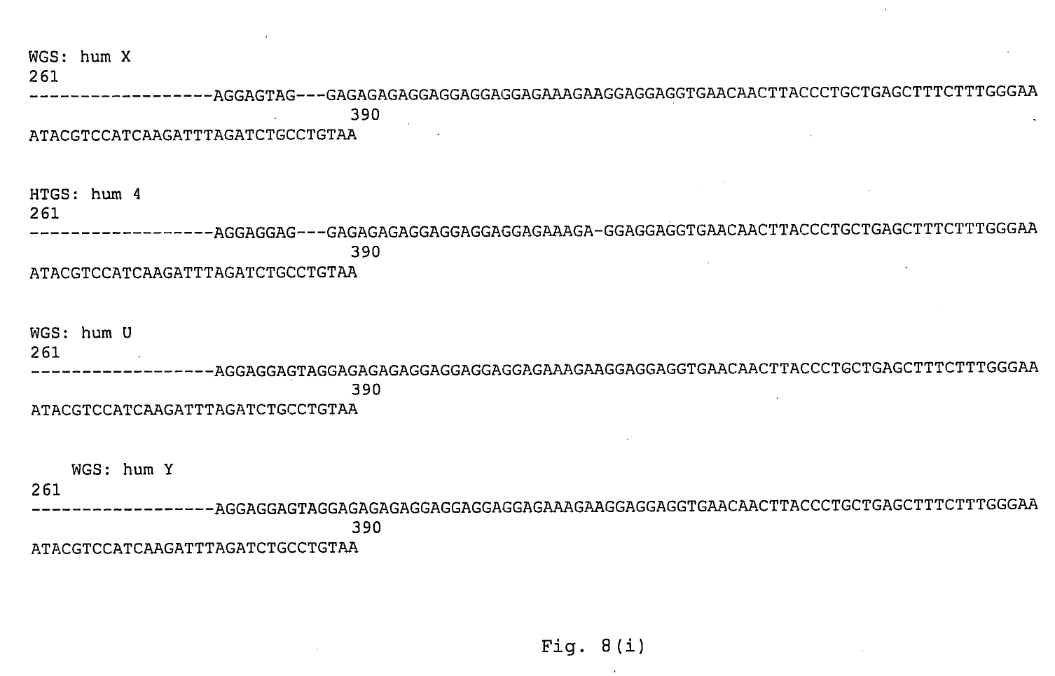

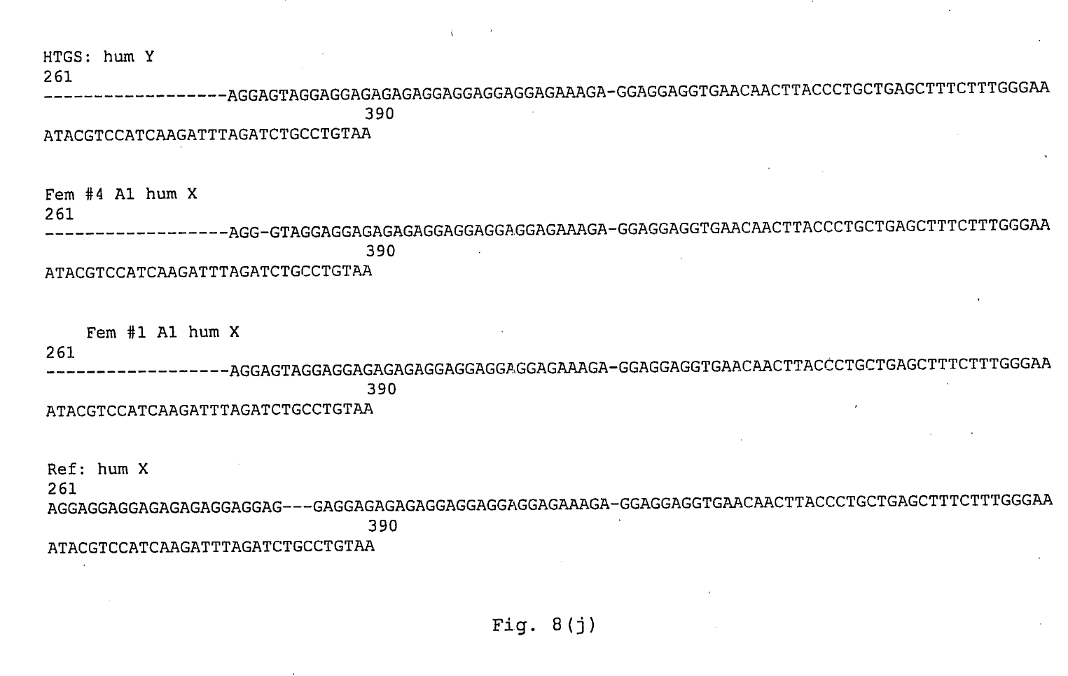

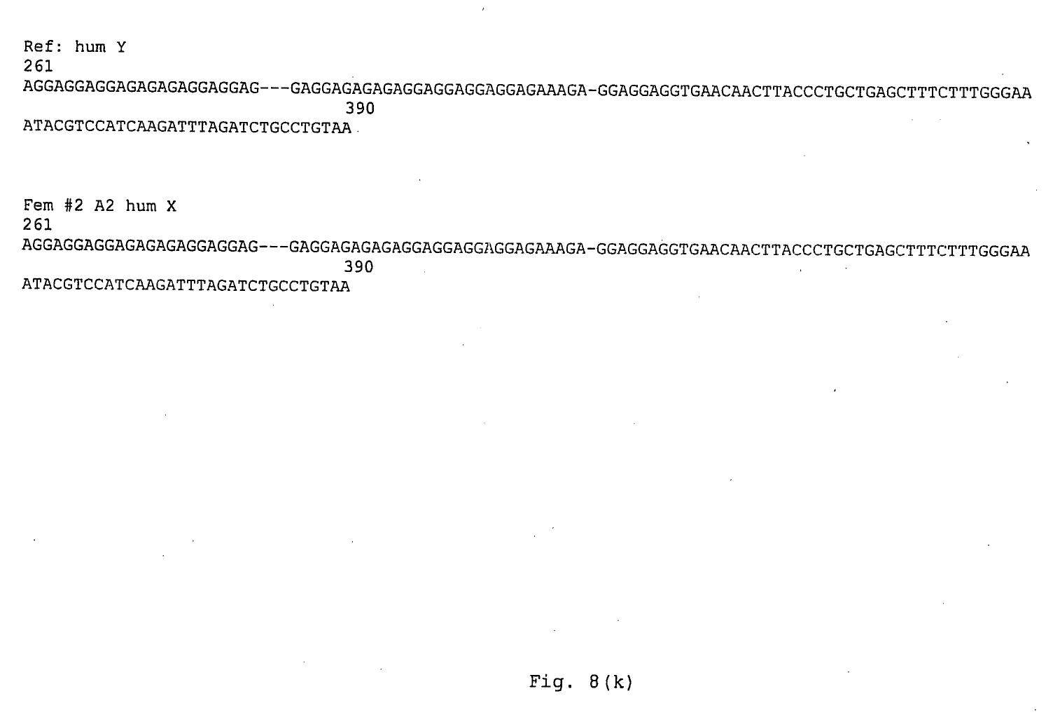

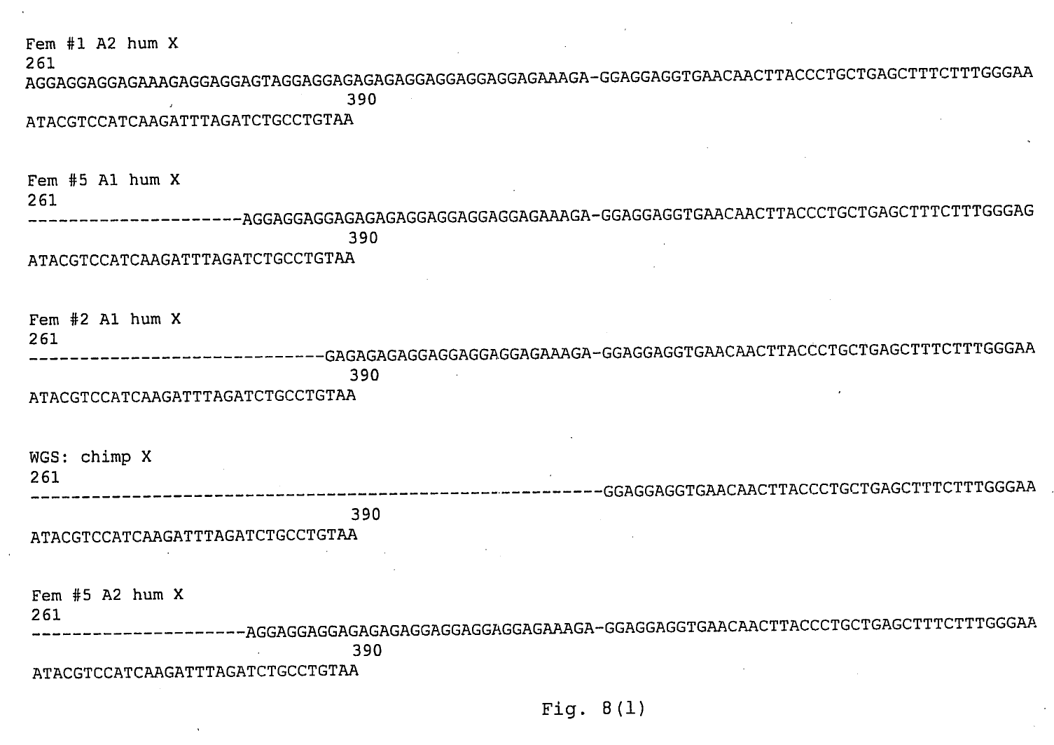

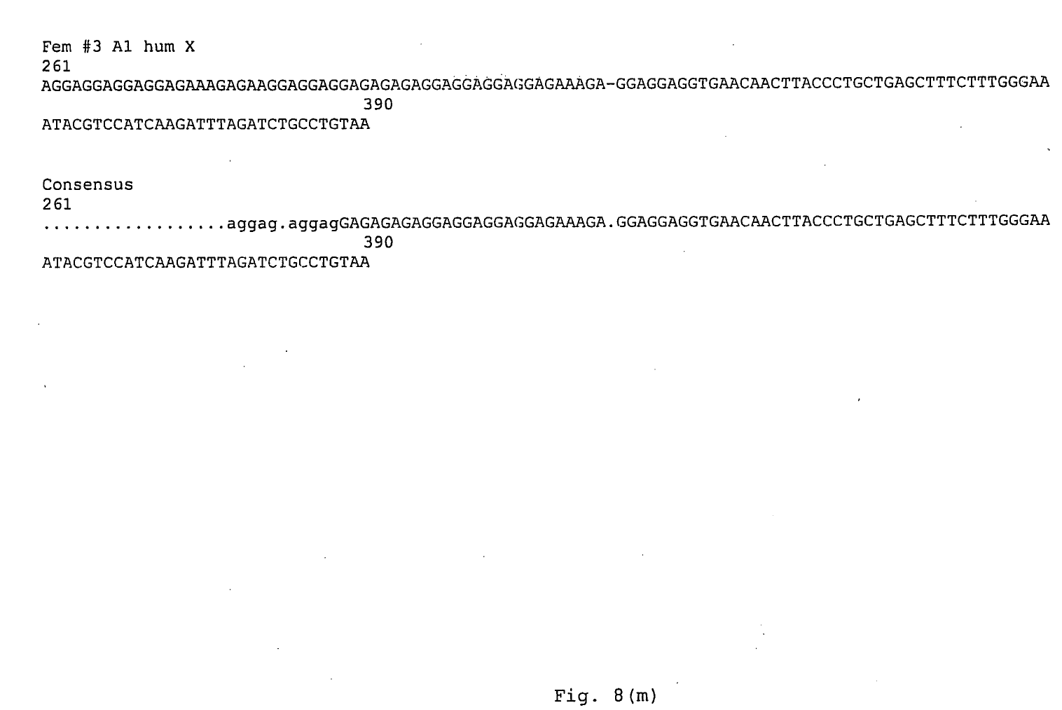

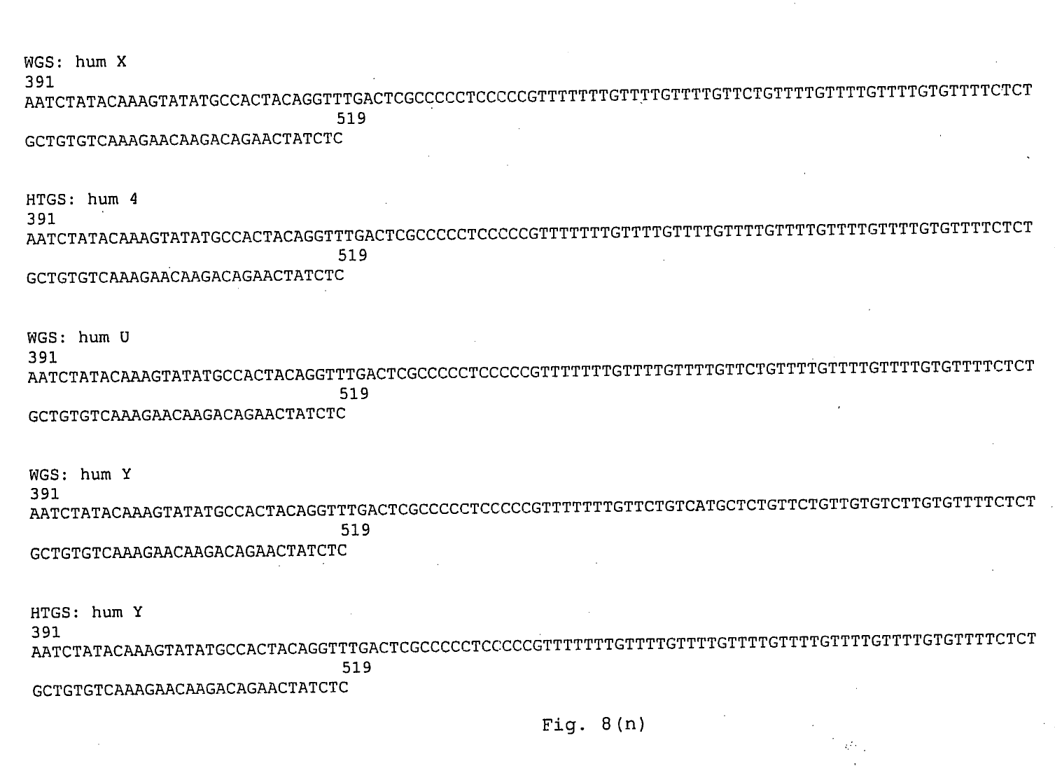

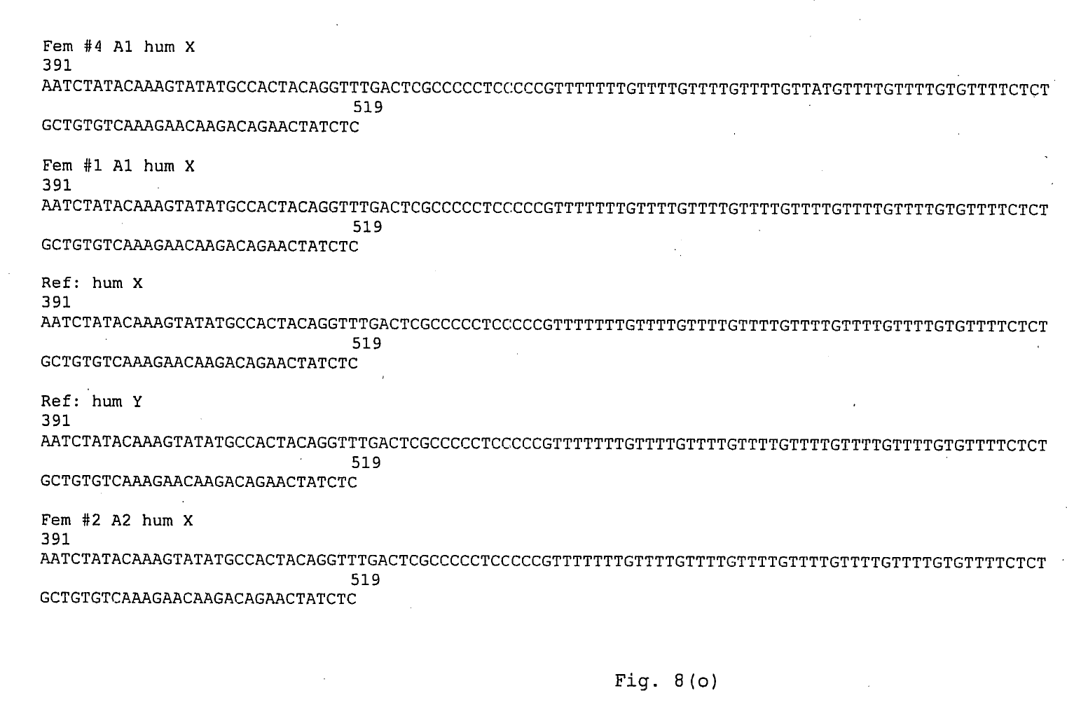

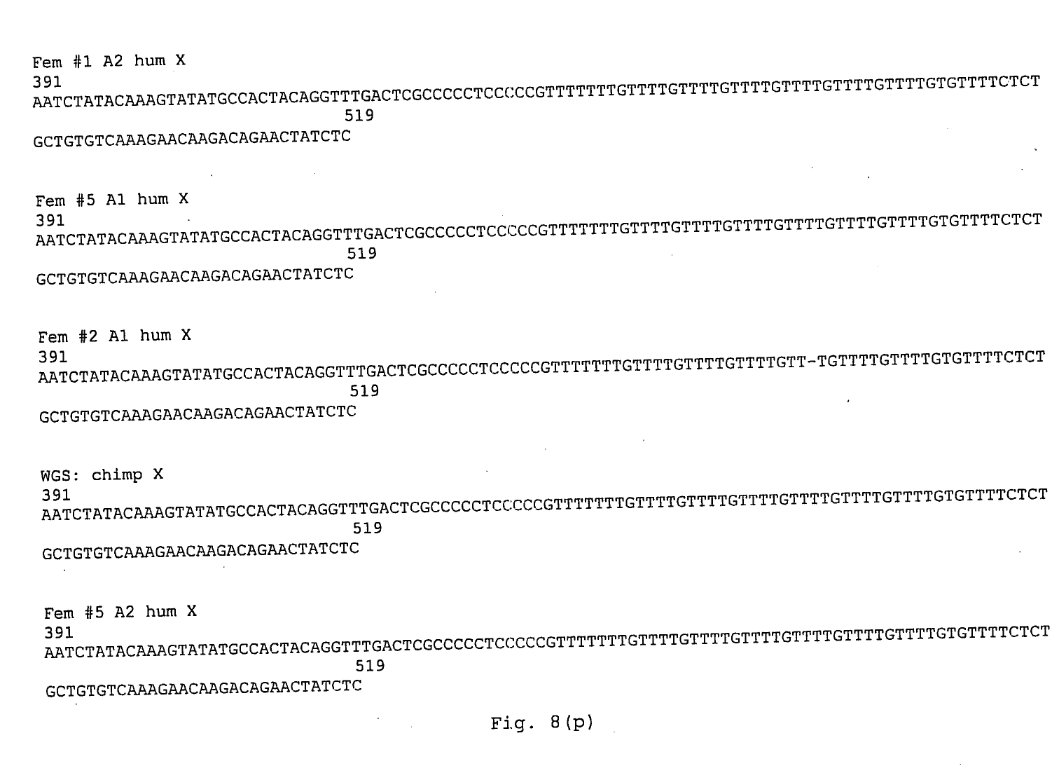

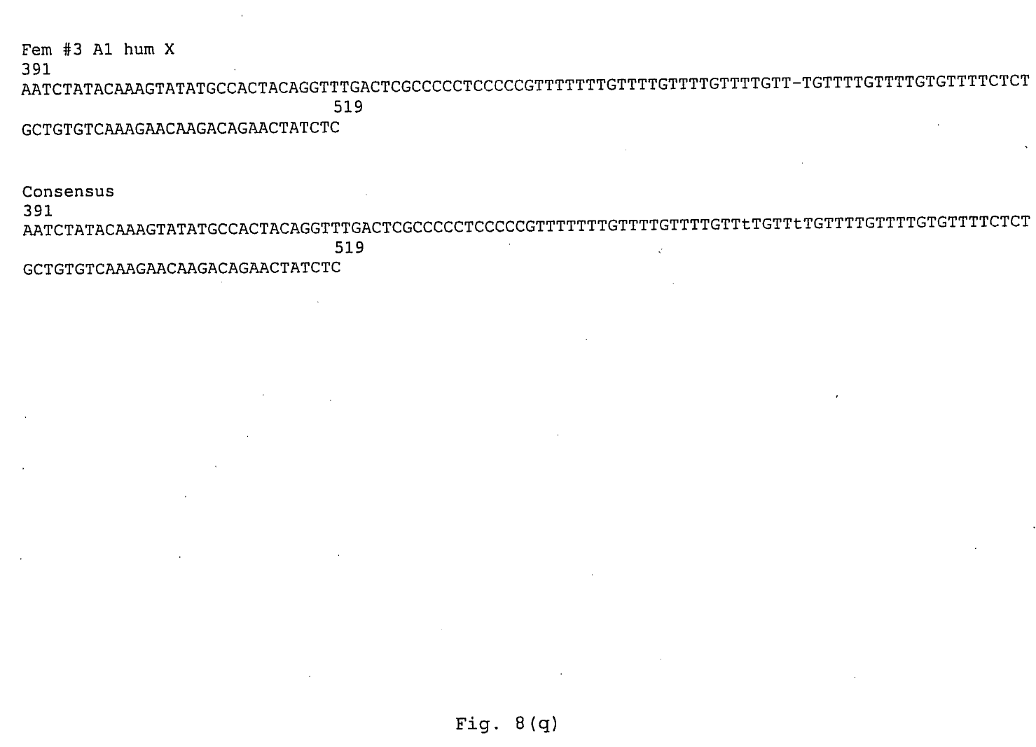

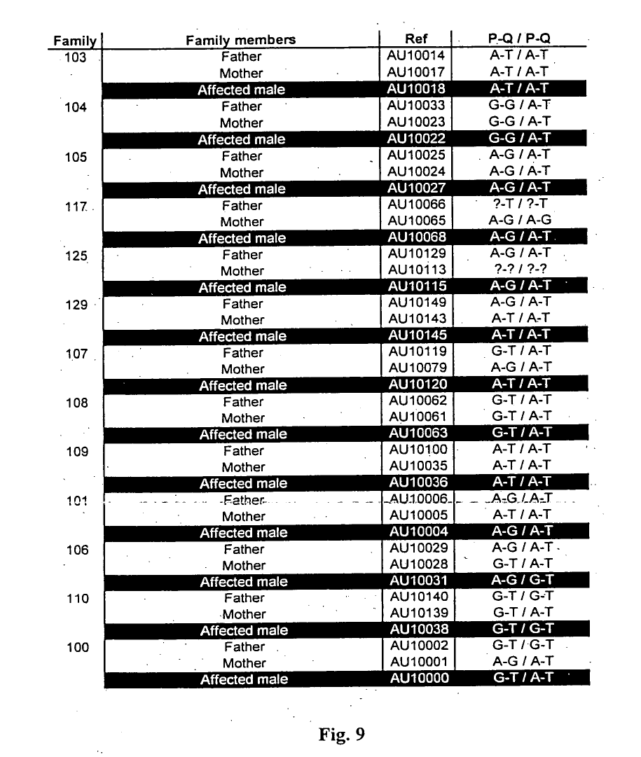

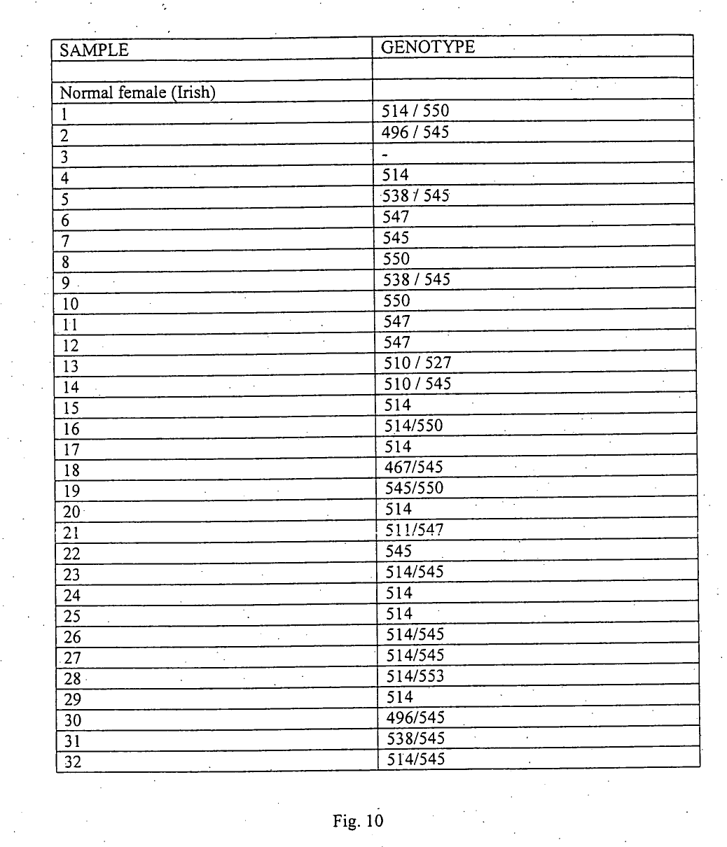

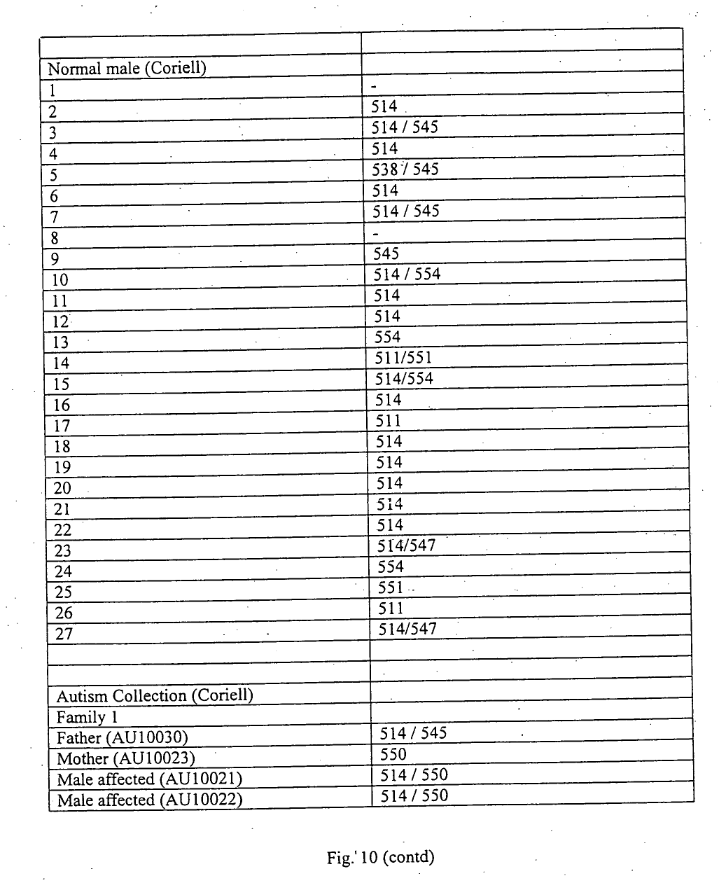

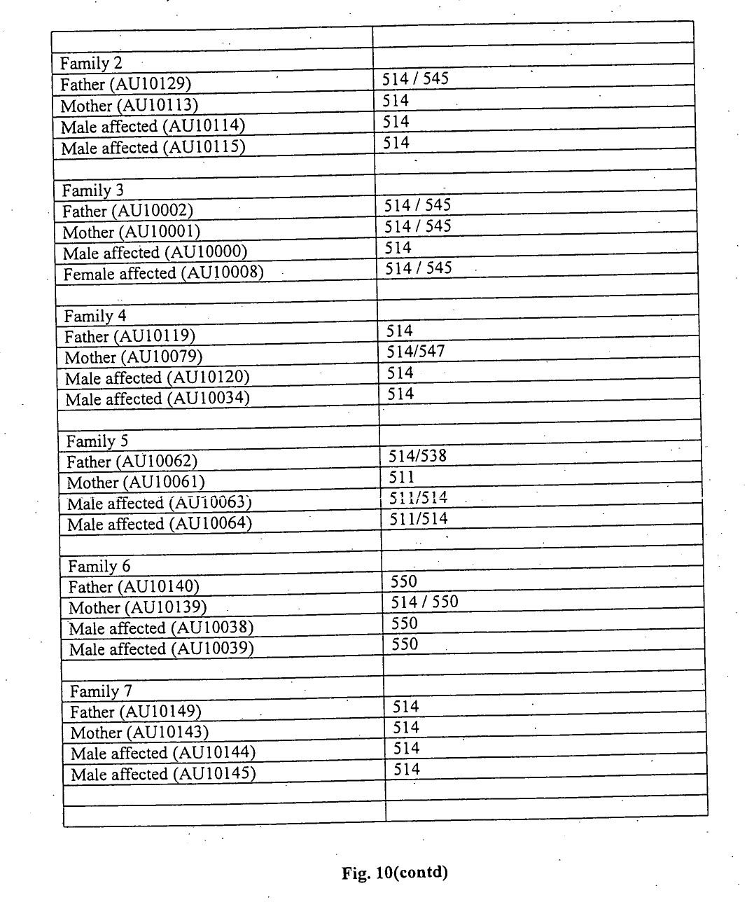

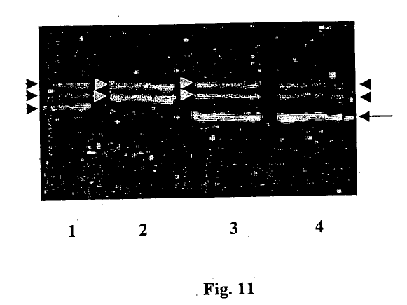

The Xq/Yq PAR as shown in FIG. 1 exhibits an unusual form of genetic/epigenetic regulation (Ciccodicola et al., 2000). The PAR consists of approximately 300 Kb and contains the HSPRY3, AMD2 (S-AdometDC-like), SYBL1, TRPC6-like, IL9R and CXYorf1 genes. X chromosome-specific genes adjacent to the PAR include the TMLHE, CLIC2, RAB39B, Histone H2 family, member B homologue (H2AFB), LOC401622 (LINE-1 reverse transcriptase homologue); LOC401623 (LINE-1 reverse transcriptase homologue), C6.1A, Mature T-cell proliferation 1 (MTCP1), Hepatitis C virus core-binding protein 6 (HCBP6) and VBP1 genes. HSPRY3 and SYBL1 undergo random X-inactivation in females, but preferential Y-inactivation in males. IL9R is expressed from both alleles in both males and females i.e. behaves in a standard pseudoautosomal manner. The expression patterns of the parental alleles of the AMD2 and TRPC6-like genes are unknown. AMD2 and TRPC6-like may be non-expressed pseudogenes.

Recessive ASD susceptibility alleles, or deregulation of X-linked copies of Y-inactivated, or X-specific, genes would therefore be exposed in males, explaining the increased incidence of the condition in males. Also, for some conditions, loss of imprinting leading to over-expression of Y-inactivated genes may occur. Alternatively, there may be environmental or modifier gene-mediated epigenetic deregulation of the region, with similar (or more unpredictable) patterns of inheritance. In principle, therefore, this region can explain male-biased affected sex ratios, with scope for further complexities due to deregulation of the epigenetic mechanisms that operate across the region. There are strong precedents for combinations of cytogenetic/epigenetic abnormalities in imprinted Beckwith-Wiedemann and Angelman/Prader-Willi syndromes. Such complexities would probably confound standard genetic marker linkage analyses in families.

There may also be interactions between homologous genes on different chromosomes in the parental germline, embryonic tissues, or postnatally, which may affect their epigenetic regulation and expression characteristics.

Other conditions such as attention deficit hyperactivity disorder (ADHD), tuberous sclerosis (TS), and clear cell carcinoma of the kidney (CCCK) may also be identified and diagnosed using the ASD locus. ADHD also has male biased sex ratios. TS is associated with a high rate of autism and increased rate of CCCK.

Details and proposed relevance of genes in the region.

The HSPRY3 gene (sequence accession: AJ271735) is a homologue of Drosophila Sprouty, which is involved in specifying forebrain/hindbrain developmental patterning. Chick Sprouty is expressed at the isthmus and rhombomere 1 (which gives rise to the entire cerebellum). Sprouty inhibits FGF8, which is also implicated in hindbrain patterning via inhibition of Hox gene expression. Mouse Sprouty genes are also expressed at the isthmus. A key finding in brain scans in autistic subjects is increased cerebral volume coupled with cerebellar abnormalities.

The SYBL1 gene (sequence accession: AJ271736) encodes a synaptobrevin-like protein (TI-VAMP/VAMP-7), a member of the SNARE protein family that includes synaptobrevin, syntaxin and SNAP-25, and has wide involvement in cellular secretion mechanisms. SNARE complexes are integral to synapse function and, in the gastrointestinal tract, in exocytosis and gastric parietal cell function. The SNAP-25 protein has been associated with attention deficit hyperactivity disorder (ADHD) (Brophy et al., 2002)—a condition which also exhibits a male-biased sex ratio among affected individuals, and which may be classed as part of the wider autistic spectrum. SYBL1 and SNAP-25 encoded proteins may interact biochemically to influence neurite outgrowth (Martinez-Arca et al., 2000). Suggestive similarities between aspects of some autism cases and tetanus, including 4:1 affected sex ratio have been observed (Bolte 1998). Bolte proposes that some autism cases are caused by gut infections with Clostridia spp. However, a more viable hypothesis is that SYBL1 is a susceptibility locus for both tetanus and autistic spectrum disorders. (Tetanus toxin cleaves the synaptobrevin protein, which is a homologue of the protein encoded by the SYBL1 gene.)

The IL-9R gene encodes the interleukin-9 receptor, which interacts with the gamma chain of the IL-2 receptor for signalling. There is considerable functional redundancy between various Th2 cytokines, therefore any hypothesis relating aberrant IL-9R regulation to immune abnormalities found in autism must be considered speculative. Literature exists on immune abnormalities in autism, including a recent report of possible autoimmune enteropathy (Torrente et al., 2002). Autism is associated with increased serum IgE. IL-9 is strongly implicated in the pathophysiology of allergic diseases, with IgE overproduction (Levitt et al., 1999).

The epsilon-N-trimethyllysine hydroxylase gene (TMLHE) encodes the first enzyme (EC 1.14.11.8) in the carnitine biosynthetic pathway. It converts epsilon-N-trimethyllysine to beta-hydroxy-N-epsilon-trimethyllysine. The other source of carnitine is the diet. Carnitine is critical for mitochondrial function. Many autistics have reduced carnitine and increased lactic acid. In addition, trimethyllysine is a key modification of histone H3 and marks active genes in Drosophila. Also, carnitine suppresses position-effect variegation (PEV) in Drosophila, and acetyl-carnitine inhibits the cytogenetic expression of the fragile X in vitro. Therefore, this pathway may have general effects on chromatin structure and gene expression/silencing. Mutations in the MECP2 gene, (the product of which binds to methylated DNA), are implicated in Rett Syndrome—a severe neurodevelopmental disorder with autistic features.

AMD2 (S-AdoMetDC-like) is related to S-adenosylmethionine decarboxylase proenzyme (AdoMetDC, SamDC). AdoMetDC is critical to polyamine biosynthesis and obtains AdoMet (i.e. S-adenosylmethionine) from the same pool as that which provides methyl donors for DNA methyltransferase enzymes. There is abundant evidence that alterations in AdoMet levels affect Drosophila and mouse PEV and gene expression/silencing through effects on DNA methylation and chromatin structure. The AMD2 locus may be a non-expressed pseudogene, or might express a non-coding RNA that influences AdoMetDC mRNA processing or translation. Alternatively, this locus might acquire de novo expression patterns following mutations in the region.

‘Similar to transient receptor potential cation channel, subfamily C, member 6’ (TRPC6-like) encodes a membrane channel. This family of channels allow Ca(2+) influx linked to phospholipase C activity. They are widely expressed, but an emerging theme is that many are predominantly expressed in the central nervous system and function in sensory physiology.

VBP1 encodes a von Hipple-Lindau (VHL) binding protein. VHL is frequently mutated in clear cell carcinoma of the kidney. Significantly, the genetically well-characterised brain disease, tuberous sclerosis (TS), is associated with a high rate of autism and increased rate of clear cell carcinoma of the kidney (CCCK) that is not associated with mutations in the gene encoding VHL. Genome instability in TS may result in deletion or deregulation of the region containing VBP1 and autism-associated genes.

RAB39B is a member of a large family of GTPases involved in vesicular trafficking. It was cloned from a human fetal brain cDNA library.

CLIC2 encodes a chloride intracellular channel of unknown function.

In addition to neuronal and brain pathology, deregulation of the region may result in pathology associated with other organ systems, due to the wide expression patterns of some of the genes. The IL9R gene has previously been implicated as a susceptibility factor in asthma. HSPRY3 is implicated in lung development and might alternatively explain increased susceptibility of some children to asthma and chest infections.

SYBL1 may be involved in a variety of secretory processes in many cells or tissues and may be the basis for reports of increased susceptibility to gastrointestinal disorders and ear infections in autistic children. The SNARE secretory complex (including synaptobrevin) is also implicated in organ of corti function and deregulation of SYBL1 might contribute to poor balance and coordination of movements in autistic individuals. There is also accumulating evidence that secretory processes in immune cells are mediated by SNARE complexes. Deregulation of SYBL1 might therefore explain altered immune responses and cytokine profiles in autism. TRPC6-like gene products may also function in immune cell physiology (Heiner et al., 2003).

The adjacent cluster of genes on Xq28 (VBP1, RAB39B, CLIC2 and TMLHE) may also be deregulated in a subset of autistics. TMLHE may have diverse indirect effects on gene regulation via chromatin structure, and also on mitochondrial function via regulation of carnitine production. This may explain hypotonia observed in autistics and a variety of other metabolic disorders such as lactic acidosis.

RAB39B, by extrapolation with other RABs, is likely to be involved in cell secretory processes (see notes on SYBL1 above). CLIC2 has an unknown function, but note that synaptic vesicle exostosis is associated with complex interactions between SNARE complexes, RAB proteins and calcium channels (Hibino et al., 2002).

Bolte (1998) noted the biased sex ratio amongst tetanus cases, which is similar to that seen in autism (4 Male: 1 Female). SYBL1 encodes a tetanus toxin insensitive paralog of synaptobrevin, the principle protein cleaved by tetanus toxin, and therefore SYBL1 may be a susceptibility or resistance (protective) locus for overt clinical tetanus. This suggests the possibility of identifying those genetically susceptible or resistant to tetanus, and may have implications for tetanus vaccination programs.

The detection and identification of the ASD locus has many applications such as use in lifestyle and education intervention, drug development, gene and cell therapies, animal models and reactivation of epigenetically silenced genes.

The detection and identification of the ASD locus has potential in the diagnosis, prognosis, prophylaxis, treatment and further research in the area of autism or related disorders.

The methods described indicate strategies for the development of rational therapies for the clinical spectrum of autism. It will allow early diagnosis and intervention for a large proportion of autistic individuals. It will allow identification of the specific genes that are deregulated in individual patients resulting in more targeted therapeutics. It will indicate a rational basis for testing of other relevant biochemical, metabolic or physiological parameters as an aid to diagnostics, and to develop and monitor novel treatment strategies.

Currently, a variety of dietary manipulations are used in therapy for individuals affected by autistic spectrum disorders including ADHD, with variable results such as B vitamin, essential fatty acid, amino acid supplementation, removal of gluten from the diet, injections of secretin etc. These treatment strategies are based on hypotheses derived from the observed clinical features across the autistic spectrum, and a large component of trial-and-error. The identity of the deregulated genes in autism will provide a more rigorous framework for determining and testing suitable therapies, derived from knowledge of the biochemical pathways, cells and organ systems in which the relevant genes are known to function. For example, deficiency of the protein encoded by SYBL1 may alter SNARE complex function in secretion of digestive enzymes. Knowledge of the identities of the enzymes that are disrupted, and the specific foods that may therefore be improperly digested and absorbed will allow rational design of dietary supplements.

Rational pharmacological interventions for autism are currently almost non-existent. The identification of the genes and associated gene regulatory and biochemical/physiological networks will facilitate targeted design of appropriate pharmacologically active agents. Specifically, agents that modify SYBL1 gene function, SYBL1 mRNA translation, SYBL1-encoded protein function, SNARE complex function, cellular secretory processes, including at neuronal synapses, neuromuscular junctions, immune cell secretory processes, digestive tract secretory processes, secretory processes in other cell or organ types. The products of other genes in the region (or the biochemical or physiological networks within which they work) may also be amenable to pharmacological modification e.g. the HSPRY3 gene product. Genes in the X chromosome-specific region may be relevant to therapy if they are deregulated.

There are a number of extant or developing technologies in the field of gene therapy. They include the delivery of genetic material, capable of expression in the recipient cell, via virus-derived or other vectors (e.g. adenovirus, lentivirus, mammalian artificial chromosomes). The genetic material may consist of a gene promoter attached to a gene open reading frame encoding a protein that is missing or mutated in an autistic individual. The genetic material may also consist of a gene promoter attached to a DNA sequence that, once transcribed, produces a catalytic RNA molecule e.g. ribozyme, siRNA, microRNA that targets a gene product (mRNA) that is deregulated in an autistic individual.

The method described herein specifies that the SYBL1 and HSPRY3 genes and their products are primary targets for such therapies. In addition some or all of the other genes in the Xq/Yq PAR or adjacent X chromosome-specific region may, in some or all autistic individuals be suitable targets for such therapeutic methods. A further aspect to this is the removal of stem cells from autistic individuals, followed by genetic modification of these cells as described above, and their reintroduction into autistic individuals. A further aspect is the removal of stem cells from unaffected relatives, or unrelated, tissue-matched individuals, and the introduction of these cells into autistic individuals.

A deduction from the method described herein is that there are genomically intact, but epigenetically silenced normal copies of some of the genes (SYBL1, HSPRY3, possibly TRPC6-like) in the region that may be reactivated by (for example) DNA demethylating agents such as 5-azacytidine or other chromatin modifying molecules.

Therefore, targeted reactivation of epigenetically silenced genes would be an important application of the invention. The key concept arising from the method described herein is that, for autistic individuals, such technologies should be targeted to genes in and adjacent to the Xq/Yq PAR, particularly SYBL1 and HSPRY3.

A further deduction from the method described herein is that there are DNA-binding proteins such as transcription factors and chromatin proteins that interact with the regulatory regions of genes in the Xq/Yq PAR and adjacent X-chromosome specific region and affect the expression of genes in the region such as SYBL1 and HSPRY3.

These include the factors listed in Tables. 1 and 2, the zinc fingers CTCF (sequence accession: AF145477, NM_{—}006565) and BORIS (sequence accession: AF336042, AL160176, NM_{—}080618), and other DNA-binding or chromatin proteins that regulate gene expression or imprinting such as the HP1 family (sequence accessions: CBX3: NM_{—}007276; CBX5: NM_{—}012117; CBX1: NM_{—}006807), DNA methyltransferases (sequence accessions: DNMT3A: NM_{—}022552, AB076659, AF503864; DNMT2 and splice variants: NM_{—}004412, AJ223333; DNMT3B and splice variants: NM_{—}006892, AL035071; DNMT1: NM_{—}001379, AC010077), and histone acetyltransferases and deacetylases. Variants of the genes encoding these proteins may be considered candidate susceptibility genes for autistic spectrum disorders.

The invention will be more fully understood by the following examples.

A variety of methods of assaying the locus of the invention may be envisaged using current state-of-the-art technologies to detect abnormalities in the structure and expression of the locus. Essentially, the types of techniques used are those that can distinguish alterations in gene copy number e.g. deletions, duplications, insertions; structural alterations of the locus not involving changes in gene copy number, but affecting gene expression e.g. translocations, inversions, conversions; minor structural changes (changes in DNA sequence) that affect gene expression e.g. point mutations in gene promoters, enhancers, silencers, boundary elements, splice sites, kozak sequences, open reading frames (stop codons and frame-shifting mutations, non-conservative amino acid changes), untranslated regions, polyadenylation signals; DNA repeat expansions, deletions or rearrangements; alterations in the epigenetic regulation of genes or regulatory sequences in the region, resulting in changes in chromatin structure e.g. DNA demethylation or hypermethylation, post-translational modifications of histones and non-histone proteins bound to DNA in the region, higher order packaging of DNA as euchromatin or heterochromatin, telomere structure influencing telomere stability, or spreading of telomeric heterochromatin to genes in the region (telomeric silencing); spreading of heterochromatin from the Y chromosome-specific region to the Y-linked PAR.

The genomic alterations described above may occur in, or influence the function of, any part of the genes in the region, including promoters, introns, exons, or any other regulatory motifs or regions that influence gene expression.

The biological samples used will typically be a blood sample from a normal, or developmentally (behaviourally) retarded or afflicted, child. However, other samples may appropriately be obtained including saliva, hair, amniotic fluid, biopsy of placental cells or preimplantation embryos, semen (from adult males), or cells from the buccal mucosa (cheek scraping or swabbing), tissue. The primary aim is to obtain sufficient cells for the isolation of DNA, RNA, protein or chromatin for analysis.

The mutational mechanisms may occur by a variety of different mechanisms including (but not exclusively) point mutations in gene regulatory motifs, gene conversions, gene deletions, other gene rearrangements, alterations in chromatin structure. However, the data showing loss-of-heterozygosity of markers in the SYBL1 gene region (FIGS. 2, 4, 5) indicates that a major mechanism of causation of autistic spectrum disorders is likely to be either gene conversion or gene deletion spanning the SYBL1 locus. In addition, the data concerning the variation in repeat structure in the HSPRY3 promoter region implicates polymorphisms in the ‘MER31I c’ or ‘GTTTT’ repeats, or their binding proteins, in the causation of autistic spectrum disorders (FIGS. 8, 9, 10, 11, Tables 1 & 2).

Preferred diagnostic methods used are those that detect gene conversion or gene deletion events. Such methods include those based on technologies such as cloning and sequencing of DNA from the region; quantitative (e.g. Taqman or real-time) polymerase chain reaction (PCR); ‘long’ PCR across deletion boundaries; restriction enzyme cleavage, Southern blotting and hybridisation of DNA probes from the region; in situ hybridisation to DNA, chromosomes, or cells using DNA probes from the region; DNA ‘array’ or ‘chip’ technologies containing DNA from the region, and hybridised with sample DNA; DNA methylation analysis using methylation-sensitive restriction enzyme cleavage, Southern blotting and hybridisation of probes from the region; DNA methylation analysis by bisulphite treatment of sample DNA followed by cloning and sequencing of PCR products from the region, or variations of this technique using PCR primers capable of amplifying sequences derived from methylated or unmethylated DNA; analysis of chromatin structure using DNA nuclease digestion of chromatin, followed by Southern blotting and hybridisation of probes from the region; genetic studies in extended families using polymorphic microsatellite markers in the region.

In addition, abnormal regulation of genes in the region may be detected by gene expression studies on tissue samples. These methods require, as a starting point, the isolation of total RNA, mRNA, or protein from samples. A variety of standard techniques may be applied including: quantitative northern blotting of RNA followed by hydridisation with DNA or RNA probes from the expressed (exonic) sequences in the region; quantitative reverse transcription-polymerase chain reaction (RT-PCR) using Taqman or real-time platforms; DNA ‘array’ or ‘chip’ technologies containing expressed (exonic) DNA sequences from the region, and hybridised with sample RNA or cDNA (complementary DNA); in situ hybridisation to RNA in cells using exonic probes from the region; analysis of gene expression at the protein level: western blotting of homogenised tissue, and quantification of protein using specific antibodies to proteins encoded by genes in the region; use of specific antibodies to quantify proteins encoded by genes in the region in an ELISA or related format; ‘array’ or ‘chip’ technologies using specific antibodies to quantify proteins encoded by genes in the region; immunohistochemistry of histological tissue sections or cells attached to glass slides, using specific antibodies to quantify proteins encoded by genes in the region.

The genes in this region are highly conserved amongst mammals. The techniques outlined herein may be relevant to the identification or production of mutants (e.g. mouse mutants) with autism, for further research into mechanisms of pathology, and therapeutics.

Although the genes in the region are conserved in mammals (these are referred to as ‘orthologues’ or ‘homologues’), linkage of the genes (including Y chromosome linkage) is not conserved, even in the higher primates (apes), and is not found in, for example, rodents, where the genes are distributed on the X chromosome and autosomes. However, mouse models of autistic spectrum disorders may be produced by gene targeting of the genomically dispersed orthologs in the mouse, followed by mouse breeding programs to produce mice with deregulated expression of the relevant genes, or their paralogues. Human artificial chromosomes or bacterial artificial chromosomes containing part or all of the human Xq/Yq PAR may also be used to study mutational mechanisms and to produce cellular (cells cultured in vitro) or mouse models of aspects of the disorder.

Antibodies may be prepared by methods commonly known in the art which specifically bind to an epitope of an altered marker encoded by genes in the Xq/Yq pseudoautosomal (PAR) region and adjacent chromosome-specific (Xq28) region. Antibodies may also be prepared which specifically bind to an epitope of an altered marker encoded by genes (listed in tables 1 and 2) that regulate genes in the Xq/Yq pseudoautosomal (PAR) region and adjacent chromosome-specific (Xq28) region.

Also envisaged within the scope of the invention are assay kits based on the identification of the ASD locus. The kits may be used for screening for an alteration in the genetic or epigenetic markers associated with autism or related disorders comprising an antibody as described above or a probe or primer selected from any one or more of SEQ ID Nos 1 to 13 and 35 to 41. Reagents suitable for western blot, immunohistochemcial assays and ELISA assays are those which are commonly known in the art.

All of the above techniques, or variations thereof, are well known in the field.

### EXAMPLES

### Example 1

Origin of DNA Samples from Families with Autism (Normal and Affected Individuals) (See http://coriell.umdnj.edu/).

DNA samples were obtained from the United States Coriell Cell Repository (CCR) Autism Resource comprising a collection of nineteen families, which in addition to probands, includes some or all of the following: affected and non-affected siblings, parents and grandparents. Unrelated controls were obtained from the CCR/National Institute of Aging Longevity Collection, and consisted of healthy young adults, and from the CCR/National Institute of General Medical Sciences Caucasian Panel (HD200CAU). FIG. 2 lists the different families examined from the Autism Collection; FIG. 4 lists Control samples. For the analysis of the HSPRY3 promoter region, an additional set of thirty two samples from normal young females of Irish origin, collected under the auspices of a Reproductive Tissue Bank, were analysed.

### Example 2

Identification of Polymorphic Genetic Markers in the Xq/Yq PAR and Adjacent X Chromosome-specific Region.

Public DNA sequence databases were scanned for polymorphisms in the region that would allow restriction enzyme fragment length polymorphisms (RFLPs) to be developed for genetic studies. PCR primers spanning the polymorphic site were developed for amplification of short PCR products from genomic DNA, as shown in FIG. 3. PCR products were digested with the appropriate restriction enzyme and the resultant digestion products were analysed by agarose gel electrophoresis. Each sample genotype was scored as +/+, +/−, or −/−, depending on whether a digestion product was present (+) or absent (−) (FIGS. 2, 3, 4). Additional polymorphic genetic markers were developed by scanning the DNA sequence of genomic contig NT_{—}025307.13 for short tandem (dinucleotide) repeats (STRs), which are likely to provide additional polymorphisms for genetic studies. Identified repeats were spanned with PCR primers as described for RFLPs above, and products were analysed using an ABI-310 instrument (FIG. 3). The allelic sequence structure of STRs were determined in control and affected populations for STR#3, STR#5, STR#9, STR#10, SYBL1-STR#1B and SYBL1-STR#2B. For the analysis of the ‘MER31I c’ and ‘GTTTT’ repeats in the HSPRY3 promoter region PCR primers spanning the repeats were designed (FIG. 3) and PCR products were analysed by agarose gel electrophoresis (FIG. 11), ABI-310 capillary electrophoresis (FIG. 10), and cloning and sequencing (FIG. 8).

### Example 3

Detection of Association of Loss-of-Heterozygosity (LOH) at the SYBL1 Locus with Autism.

The entire CCR Autism Collection was genotyped for the following markers in the Xq/Yq PAR: SYBL1-XhoI, SYBL1-STR#1B, SYBL1-STR#2B, LH1, RsaI, StyI (FIGS. 1, 2, 4). In addition, the RsaI marker was applied to the Control samples listed in Example 1 (FIG. 4).

Controls were:

Published genotype frequencies from the public databases for RsaI (http://www.ncbi.nlm.nih.gov/SNP/snp_ref.cgi?rs=1883051), which were similar to those found in our experiments on the Control samples listed in Example 1.

Parents/unaffected family members. All listed markers between SYBL1-XhoI and StyI (FIGS. 1, 2, 4) were applied to grandparents, parents and siblings comprising eighteen fathers, nineteen mothers, and nineteen other unaffected family members (fifty six unaffected family members in total).

The RsaI marker was applied to the entire HD100CAU caucasian panel comprising two hundred individuals, of which one hundred and ninety nine were successfully genotyped (FIG. 4).

The SYBL1-XhoI, SYBL1-STR#1B, SYBL1-STR#2B, LH1, RsaI, and StyI markers were applied to twenty males and twenty females (forty individuals in total) under forty years of age from the CCR Longevity Panel (see Example 1), of which between nineteen and twenty individuals of each sex were successfully genotyped for each marker.

For those samples obtained from the nineteen families from the CCR Autism Resource, statistically significant differences between the distribution of homozygotes and heterozygotes were detected between the affected groups and the unaffected groups for markers in the SYBL1 region, suggesting the occurrence either of i. a susceptibility allele; ii. a gene conversion event; or, iii. a gene deletion. The ratio of homozygotes to heterozygotes for the proximal XhoI marker in the SYBL1 gene promoter region, and the distal StyI marker in the IL9R gene, was not significantly different between affected and unaffected groups suggesting that the region predominantly affected in autistic individuals lies between these two markers (FIG. 5). However, the genomic area affected by LOH may extend beyond these markers in a subset of individuals. The Fisher's Exact 2-tailed P values (FIG. 5) indicate that the observation of LOH in this region is not due to chance, but, rather, reflects a causative relationship between this genomic region and autism and related disorders.

A further control is derived from a comparison of the genotype frequencies of polymorphic markers in the various control groups (Longevity and Caucasian panels) with the affected and unaffected groups from the nineteen families from the Autism Resource. In all available comparisons, the distributions of homozygotes and heterozygotes in the two unaffected groups (derived from the Longevity and Caucasian panels) are not significantly different to unaffected individuals from the Autism Resource (FIGS. 6, 7). However, both unaffected groups (Longevity and Caucasian panels) are significantly different from at least one of the affected groups derived from the Autism Resource panel (‘index cases’ and ‘all affected’), for markers in the SYBL1 genomic region (FIGS. 6, 7). This indicates that the control (unaffected) group derived from the Autism Resource is not unusual, and that these individuals are similar in genetic structure to unrelated controls from the general population. It also indicates that the affected groups from the Autism Resource are significantly different to unrelated controls from the general population.

Note that in all comparisons P≦0.05 is considered to be statistically significant.

### Example 4

Detection of extensive variation in ‘MER31I c’ and ‘GTTTT’ repeat sequences in the HSPRY3 gene promoter region.

The public databases were scanned for chimpanzee (Pan troglodytes) and human DNA sequences in the SPRY3 gene promoter region, and multiple alignments of the sequences were carried out (FIG. 8).

FIG. 8 shows the multialignment (Corpet, 1988) of genomic DNA sequences encompassing two major repeats within the human (hum) and chimpanzee (chimp) SPRY3 promoter regions (equivalent to nucleotides 510246-510738 in RefSeq chromosome X contig NT_{—}025307.13 and nucleotides 66107-66599 in RefSeq chromosome Y contig NT_{—}079585.2). The sequences are derived from a combination of sources: National Center for Biotechnology information (NCBI: www.ncbi.nlm.nih.gov) databases including the whole genome shotgun (WGS) database, the high-throughput genomic sequencing (HTGS) database and the reference sequence project (RefSeq) database, plus cloned and sequenced PCR products from genomic DNA derived from five female (Fem) subjects (where two alleles were observed they are represented as A1 and A2). Source database sequence identifiers are as follows (the suffixes ‘X’, ‘Y’ and ‘4’ represent chromosome number, whereas ‘U’ represents unmapped sequence): AADC01149041.1 (WGS: hum X), AADB01164924.1 (WGS: hum U), AADC01160617.1 (WGS: hum Y), AADA01175381.1 (WGS: chimp X), AC009620.4 (HTGS: hum 4), NT_{—}025307.13 (RefSeq: hum X), NT_{—}079585.2 (RefSeq: hum Y). AC025226.4 (HTGS: hum Y).

The ‘GTTTT’ DNA repeat appears to be not as variable as the ‘MER31I c’ repeat. However, sample Fem #3 A1 has one variant sequence ‘GTTT’. Sample WGS: hum X has one variant sequence ‘GTTCT’. Sample WGS: hum Y has the variant sequence ‘GTTCT/GTCAT/GCTCT/GTTCT/GTTGT/GTCTT’.

Sequences from Fem #1, 2, 3, 4, 5 are single pass sequences which may contain minor uncorrected errors. However, these sequences establish the variability of the ‘MER31I c’ and ‘GTTTT’ repeats in the human population, which may be of functional biological or pathological significance.

This analysis identified the ‘MER31I c’ repeat in the human HSPRY3 gene promoter as having undergone considerable expansion compared to the chimpanzee sequence. Further public human clones of putative X and Y chromosome DNA sequences exhibited variations of the ‘MER31I c’ and ‘GTTTT’ repeats. Particularly, noteworthy is a variant Y chromosome sequence containing multiple mutations in the ‘GTTTT’ repeat that abolishes SRY binding sites and adds a Progesterone receptor binding site (FIG. 8 & Table 2).

The public databases contain several human sequences that are ascribed to chromosomes other than the X and Y (FIG. 8). Such duplicated regions would potentially confound genomic and genetic analysis of the region. However, below we provide evidence that contradicts the presence of autosomal duplications of this region.

PCR primers were designed to flank the ‘MER31I c’ and ‘GTTTT’ repeats of the HSPRY3 gene promoter region (FIG. 3). PCR products were analysed by agarose gel (FIG. 11) and capillary (ABI-310) electrophoresis (FIG. 10), and by cloning and sequencing PCR products (FIG. 8). DNA sequences were obtained from five unaffected young women of Irish origin and single pass sequences are listed in FIG. 8. These sequences indicate that the majority of alleles (PCR product length polymorphisms) at this locus are likely to be due to variants of the ‘MER31I c’ repeat region.

An analysis of the HSPRY3 gene promoter region by ABI-310 capillary electrophoresis was carried out in thirty two unaffected young women of Irish origin and identified between ten and thirteen alleles at this locus (FIG. 10). Some of the alleles differed from one another by a single base pair and may represent the same sequence, which was misread by the ABI-310 instrument. The alleles are listed in FIG. 3.

Inspection of the genotype frequencies for normal males and females (FIG. 10) suggested the existence of a deleted or variant allele that does not amplify using the PCR primers used in this analysis. This is because the large number of alleles in the population predicts that homozygotes should be relatively rare. However, fourteen of thirty one females were homozygous, and seventeen of twenty five males were homozygous.

The size determination of alleles in FIG. 10 may have minor errors. For example, allele pairs 510 and 511, 550 and 551, 553 and 554 may represent three, not six, different alleles. Full description, validation and discrimination of all alleles will require extensive DNA sequencing. In the normal female population of Irish origin there are therefore potentially between ten and thirteen different alleles: 467, 496, 510, 511, 514, 527, 538, 545, 547, 550, 551, 553, 554. The high number of homozygotes (14 of 31 samples) suggests that there may be another allele that contains a deletion or other rearrangement or mutation of the region encompassing one of the PCR primers used to amplify the genomic DNA. In a normal young male population from the Coriell Aging Resource there are seven alleles: 511, 514, 538, 545, 547, 551, 554. All of these alleles are found in the normal female population of Irish origin. Similar to the normal female population of Irish origin, there are a high number of homozygotes (17 of 25 samples). In seven Autism families from the Coriell Resource there are six alleles: 511, 514, 538, 545, 547, 550. The inheritance of alleles within the families indicates that the 514 allele is Y-linked in six of the seven fathers. The 514 allele is also found abundantly in the normal young male population from the Coriell Ageing Resource, and less abundantly in the normal female population of Irish origin. These results indicate that: 1) There are a large number of alleles in males and females, which may produce different levels or patterns of HSPRY3 gene expression. 2) The 514 allele frequency may be increased in males due to it being over-represented on the Y chromosome. 3) There may be deleted, rearranged, or mutated variant alleles that require further characterisation.

The possible transcription factor binding sites of sequenced variant alleles were analysed (Table 1 and 2). In addition, proteins or regulatory RNA molecules that regulate chromatin structure, dosage compensation or genomic imprinting (e.g. heterochromatin proteins such as HP1 and homologues, and the zinc finger proteins CTCF and BORIS) may be implicated in regulating different allelic variants such that expression levels, or temporal or spatial patterns of gene expression are altered. Moreover, interactions between DNA repeats on grandmaternally and grandpaternally derived homologues in the germline, or maternally and paternally derived homologues in the embryo may epigenetically modulate HSPRY3 gene silencing or expression. An X-linked deleted variant of the HSPRY3 gene promoter may lead to a null phenotype in males (in which the Y-linked homologue is thought to be silenced), or may lead to reactivation of the Y-linked homologue, analogous to the reactivation of the paternally derived X chromosome in the extra-embryonic tissues of XpO (monosomic) mice.

Table 1 shows the analysis of potential transcription factor binding sites in the promoter region of the HSPRY3 gene spanning the ‘Mer31I c’ repeat. The major factors likely to bind to the repeat are MAZ (Myc-associated zinc finger protein)/Pur1/GAGA factor, Vitamin D receptor, RXR (Retinoid X receptor), Forkhead (FOXO). The number of binding sites for the various factors in a particular allele are predicted to vary depending on the number of repeat units and other polymorphismss of the HSPRY3 promoter DNA sequence. Different sequences affect the identity, number and location of transcriptional enhancer and suppressor proteins.

Table 2 shows the analysis of potential transcription factor binding sites in the promoter region of the HSPRY3 gene spanning the ‘GTTTT’ repeat. The major factor likely to bind to the repeat is SRY (Sex determining region Y gene product). (Other factors are listed in Table 2). The number of binding sites for the various factors in a particular allele are predicted to vary depending on the number of repeat units and other variations of the HSPRY3 promoter DNA sequence. In particular, mutations in the WGS: Hum Y sequence (FIG. 8) abolishes the SRY binding sites and adds a Progesterone receptor binding site. Different sequences affect the identity, number and location of transcriptional enhancer and suppressor proteins.

The sequence listing for each of the transcription factors is listed in Tables 1 and 2. The sequences can be supplied in the WIPO Standard ST25 if required.

Allelic variants of factors that regulate the HSPRY3 promoter or dosage compensation may be implicated in the causation of autistic spectrum disorders. MAZ/GAGA factor homologues regulate gene dosage and X chromosome dosage compensation in Drosophila. SRY variants may explain the postulated link between testosterone levels, altered digit lengths and masculinization of the brain as postulated by Baron-Cohen. The Progesterone receptor and Vitamin D receptors are expressed in the male brain and variants may influence HSPRY3 gene expression. The FOXO gene product predicted to bind to the HSPRY3 gene promoter region is homologous to the FOXP2 gene implicated in autism and language disorders on chromosome 7q31.

### Example 5

Detection of possible complete or partial Y chromosome-linkage of the 514 allele of the HSPRY3 promoter region.

A similar analysis of twenty seven unaffected young males from the Coriell Aging Resource yielded no new alleles spanning the HSPRY3 promoter ‘MER31I c’ or ‘GTTTT’ repeats but indicated a possible enrichment of the 514 allele in males, which could suggest Y-linkage or partial Y-linkage (because this allele was also seen in females). An alternative interpretation consistent with full Y-linkage is that there are two different 514 alleles with different evolutionary histories.

The presence of only seven different alleles in the normal males compared to up to thirteen in the normal females may also be consistent with Y-linkage of the 514 allele because only one X chromosome occurs in males, compared to two in females, therefore males would be expected to exhibit approximately half the variation seen in females, as we observe.

A similar analysis of seven families from the Coriell Autism Resource further suggested Y-linkage of the 514 allele because six of seven fathers had the 514 allele on their Y chromosome.

We note that five of the seven mothers of autistic children in these families carried the 514 allele suggesting a possible enrichment of this allele, or a pathological variant of 514, in the mothers of autistic individuals. One possibility is that mothers of autistic individuals carry X-linked alleles that recently recombined from a Y chromosome e.g. in their fathers' germlines.

### Example 6

Detection of a probable recombination hotspot in a small interval between the end of the HSPRY3 coding region (CDS) and the 3′ untranslated region (UTR).

A recombination hotspot is defined as a region of the genome that experiences a relatively high rate of genetic recombination relative to other regions of the genome.

The HSPRY3-SNP and HSPRY3-HinfI markers are separated by 156 bp at the distal end of the HSPRY3 coding sequence/3′ UTR region (FIG. 3). Markers that are physically contiguous are usually found to be in linkage disequilibrium. However, PCR and single pass sequencing of both parents and one affected individual from thirteen families from the Coriell Autism Resource found that all four recombination products are observed (FIG. 9), suggesting the presence of a recombination hotspot in this region.

The presence of a recombination hotspot distal to the HSPRY3 gene promoter is consistent with the possible finding of partial Y-linkage of HSPRY3 gene promoter allelic variants described above.

This region is also close to the site of origin of a transcript cloned from a human amygdala cDNA library (cDNA FLJ37291, ACCESSION: AK094610). This transcript may have a regulatory function in HSPRY3 expression in the brain, or more specifically the amygdala—a brain region strongly implicated in the aetiology of autism—and may, for example, represent the site of a tissue-specific enhancer, silencer or boundary, which may be mutated or deregulated in autistic individuals.

### Example 7

Detection of a novel mutation in the promoter region of the HSPRY3 gene in a family from the Coriell Autism Resource

Several families from the Coriell Autism Resource were analysed by PCR of the HSPRY3 promoter using primers listed in FIG. 3. Products were run on agarose gels.

In Family 104 the affected male siblings have a PCR product not found in either parent suggesting a de novo mutation in the HSPRY3 gene promoter region. This observation directly implicates mutation of the HSPRY3 gene in autism.

FIG. 11 shows the PCR analysis using primers (FIG. 3) spanning the ‘MER31I c’ and ‘GTTTT’ repeats of the HSPRY3 gene promoter. Samples: genomic DNA from Coriell Autism Resource Family 104 (samples AU10033, AU10023, AU10021, AU10022) comprising father (1), mother (2), male proband (3), affected male sib (4).

Arrowheads indicate PCR products for clarity. Arrow indicates novel PCR product in affected males, not found in parents, suggesting a novel mutation in the HSPRY3 promoter region. Note: The three bands (PCR products) observed in many samples from normal males and females and affected individuals on agarose gel electrophoresis could suggest the existence of a genomic duplication of the region on the X, Y or other chromosome in some or all individuals, as also suggested by the public genome databases HTGS: hum 4 clone. However, for all samples analysed by capillary electrophoresis, a maximum of two bands was detected suggesting that there is not a duplication of this region in the genome. (In the family shown above, the genotypes as determined by capillary gel electrophoresis were 1) Father, AU10033, 514/545; 2) Mother, AU10023, 550/550 or 550/deleted variant; 3) Proband, AU10021, 514/550; 4) Affected sib, AU10022, 514/550. The extra bands observed in agarose gel electrophoresis may therefore be due to conformational variants of the PCR products possibly generated by the ‘MER31I c’or ‘GTTTT’ repeats. These putative conformational variants were reproduced robustly in different experiments and using different PCR primer sets spanning the region (data not shown). These variants may be analogous to the variant bands detected by single-stranded conformational polymorphism (SSCP) gels, which are routinely used to detect novel mutations of unknown sequence.

As noted above, there is evidence in the public databases for duplications of this genomic region on several autosomes. The observation of three bands in three individuals from Family 104 might be taken as supportive of the presence of a genomic duplication of the region elsewhere in the genome. However, capillary electrophoresis (ABI-310) detected a maximum of two alleles per individual in this family (FIG. 11). The most likely source of the third band in the father and two affected male sibs is therefore the presence of a conformational polymorphism of the PCR product that is stable in the relatively low temperature of the agarose gel. The new band observed in the affected sibs may therefore be explained by DNA sequence variation (i.e. a mutation) affecting the conformation of the PCR product. The presence of the G-rich ‘MER31I c’ repeat may be important for generating such stable conformational variants because three bands were never observed using other PCR primers that excluded the ‘MER31I c’ repeat region.

TABLE 1

| | | | | | | | |
|---|---|---|---|---|---|---|---|
| | | | Position | | | | |
| | Further | | from- | | Core | Matrix | |
| Family/matrix | Information | Opt. | to | Str. | sim. | sim. | Sequence |

Inspecting sequence Fem#4A1humX (1-100):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGagggt |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 45-57 | (+) | 1.000 | 0.930 | gtagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 48-72 | (+) | 1.000 | 0.849 | ggaggAGAGagaggaggaggaggag |
| V$GABF/GAGA.01 | GAGA-Box | 0.78 | 50-74 | (+) | 1.000 | 0.792 | aggagAGAGaggaggaggaggagaa |
| V$RXRF/VDR_RXR.02 | VDR/RXR | 0.86 | 50-66 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 52-76 | (+) | 1.000 | 0.784 | gagagAGAGgaggaggaggagaaag |
| V$MAZF/MAZ.01 | Myc | 0.90 | 63-75 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 68-84 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 80-96 | (+) | 1.000 | 0.890 | aggaggtgAACAactta |
| | factor | | | | | | |
| | FKHRL1 | | | | | | |
| | (FOXO) | | | | | | |

Inspecting sequence HTGS: humY (1-101):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$EGRF/NGFIC.01 | Nerve | 0.80 | 42-56 | (+) | 0.768 | 0.801 | agGAGTaggaggaga |
| | growth | | | | | | |
| | factor- | | | | | | |
| | induced | | | | | | |
| | protein C | | | | | | |
| V$MAZF/MAZ.01 | Myc | 0.90 | 46-58 | (+) | 1.000 | 0.930 | gtagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 49-73 | (+) | 1.000 | 0.849 | ggaggAGAGagaggaggaggaggag |
| V$GABF/GAGA.01 | GAGA-Box | 0.78 | 51-75 | (+) | 1.000 | 0.792 | aggagAGAGaggaggaggaggagaa |
| V$RXRF/VDR_RXR.02 | VDR/RXR | 0.86 | 51-67 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 53-77 | (+) | 1.000 | 0.784 | gagagAGAGgaggaggaggagaaag |
| V$MAZF/MAZ.01 | Myc | 0.90 | 64-76 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 69-85 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 81-97 | (+) | 1.000 | 0.890 | aggaggtgAACAactta |
| | factor | | | | | | |
| | FKHRL1 | | | | | | |
| | (FOXO) | | | | | | |

Inspecting sequence HTGS: hum4 (1-98):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 28-52 | (+) | 1.000 | 0.849 | ggaggAGAGagaggaggaggaggag |
| V$GABF/GAGA.01 | GAGA-Box | 0.78 | 30-54 | (+) | 1.000 | 0.792 | aggagAGAGaggaggaggaggagag |
| V$RXRF/VDR_RXR.02 | VDR/RXR | 0.86 | 30-46 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 32-56 | (+) | 1.000 | 0.792 | gagagAGAGgaggaggaggagagag |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAcAGagaggaggaggaggag |
| V$GABF/GAGA.01 | GAGA-Box | 0.78 | 48-72 | (+) | 1.000 | 0.792 | aggagAGAGaggaggaggaggagaa |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.784 | gagagAGAGgaggaggaggagaaag |
| V$MAZF/MAZ.01 | Myc | 0.90 | 61-73 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 66-82 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 78-94 | (+) | 1.000 | 0.890 | aggaggtgAACAactta |
| | factor | | | | | | |
| | FKHRL1 | | | | | | |
| | (FOXO) | | | | | | |

Inspecting sequence Fem#1A1humX (1-101):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | heterodimer | | | | | | |
| | site | | | | | | |
| V$EGRF/NGFIC.01 | Nerve | 0.80 | 42-56 | (+) | 0.768 | 0.801 | agGAGTaggaggaga |
| | growth | | | | | | |
| | factor- | | | | | | |
| | induced | | | | | | |
| | protein C | | | | | | |
| V$MAZF/MAZ.01 | Myc | 0.90 | 46-58 | (+) | 1.000 | 0.930 | gtagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 49-73 | (+) | 1.000 | 0.849 | ggaggAGAGagaggaggaggaggag |
| V$GABF/GAGA.01 | GAGA-Box | 0.78 | 51-75 | (+) | 1.000 | 0.792 | aggagAGAGaggaggaggaggagaa |
| V$RXRF/VDR_RXR.02 | VDR/RXR | 0.86 | 51-67 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 53-77 | (+) | 1.000 | 0.784 | gagagAGAGgaggaggaggagaaag |
| V$MAZF/MAZ.01 | Myc | 0.90 | 64-76 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 69-85 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 81-97 | (+) | 1.000 | 0.890 | aggaggtgAACAactta |
| | factor | | | | | | |
| | FKHRL1 | | | | | | |
| | (FOXO) | | | | | | |

Inspecting sequence Ref: humX (1-116):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+ | ) 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagag |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.792 | gagagAGAGgaggaggaggagagag |
| V$MAZF/MAZ.01 | Myc | 0.90 | 61-73 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 64-88 | (+) | 1.000 | 0.849 | ggaggAGAGagaggaggaggaggag |
| V$GABF/GAGA.01 | GAGA-Box | 0.78 | 66-90 | (+) | 1.000 | 0.792 | aggagAGAGaggaggaggaggagaa |
| V$RXRF/VDR_RXR.02 | VDR/RXR | 0.86 | 66-82 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA/01 | GAGA-Box | 0.78 | 68-92 | (+) | 1.000 | 0.784 | gagagAGAGgaggaggaggagaaag |
| V$MAZF/MAZ.01 | Myc | 0.90 | 79-91 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 84-100 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 96-112 | (+) | 1.000 | 0.890 | aggaggtgAACAactta |
| | factor | | | | | | |
| | FKHRL1 | | | | | | |
| | (FOXO) | | | | | | |

Inspecting sequence Ref: humY (1-116):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagag |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.792 | gagagAGAGgaggaggaggagagag |
| V$MAZF/MAZ.01 | Myc | 0.90 | 61-73 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 64-88 | (+) | 1.000 | 0.849 | ggaggAGAGagaggaggaggaggag |
| V$GABF/GAGA.01 | GAGA-Box | 0.78 | 66-90 | (+) | 1.000 | 0.792 | aggagAGAGaggaggaggaggagaa |
| V$RXRF/VDR_RXR.02 | VDR/RXR | 0.86 | 66-82 | (+) | 1.000 | 0.875 | aggagagaGAGGaggag |
| | Vitamin D | | | | | | |
| | receptor | | | | | | |
| | heterodimer | | | | | | |
| | site | | | | | | |
| V$GABF/GAGA.01 | GAGA-Box | 0.78 | 68-92 | (+) | 1.000 | 0.784 | gagagAGAGgaggaggaggagaaag |
| V$MAZF/MAZ.01 | Myc | 0.90 | 79-91 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 84-100 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 96-112 | (+) | 1.000 | 0.890 | aggaggtgAACAactta |
| | factor | | | | | | |
| | FKHRL1 | | | | | | |
| | (FOXO) | | | | | | |

Inspecting sequence Fem#2A1humX (1-134):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagag |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.792 | gagagAGAGgaggaggaggagagag |
| V$MAZF/MAZ.01 | Myc | 0.90 | 61-73 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 64-88 | (+) | 1.000 | 0.849 | ggaggAGAGagaggaggaggaggag |
| V$GABF/GAGA.01 | GAGA-Box | 0.78 | 66-90 | (+) | 1.000 | 0.792 | aggagAGAGaggaggaggaggagag |
| V$RXRF/VDR_RXR.02 | VDR/RXR | 0.86 | 66-82 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 68-92 | (+) | 1.000 | 0.792 | gagagAGAGgaggaggaggagagag |
| V$MAZF/MAZ.01 | Myc | 0.90 | 79-91 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 82-106 | (+) | 1.000 | 0.849 | ggaggAGAGagaggaggaggaggag |
| V$GABF/GAGA.01 | GAGA-Box | 0.78 | 84-108 | (+) | 1.000 | 0.792 | aggagAGAGaggaggaggaggagaa |
| V$RXRF/VDR_RXR.02 | VDR/RXR | 0.86 | 84-100 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| VGABF/GAGA.01 | GAGA-Box | 0.78 | 86-100 | (+) | 1.000 | 0.784 | gagagAGAGgaggaggaggagaaag |
| V$MAZF/MAZ.01 | Myc | 0.90 | 97-109 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 102-118 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 114-130 | (+) | 1.000 | 0.890 | aggaggtgAACAactta |
| | factor | | | | | | |
| | FKHRL1 | | | | | | |
| | (FOXO) | | | | | | |

Inspecting sequence Femp#1A2humX (1-140):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.885 | aggagaaaGAGGcggag |
| | Vitamin D | | | | | | |
| | receptor | | | | | | |
| | RXR | | | | | | |
| | heterodimer | | | | | | |
| | site | | | | | | |
| V$MOKF/MOK2.01 | Ribonucleo- | 0.74 | 32-52 | (−) | 0.750 | 0.742 | ctcctcctccgccTCTTtctc |
| | protein | | | | | | |
| | associated | | | | | | |
| | zinc finger | | | | | | |
| | protein | | | | | | |
| | MOK-2 | | | | | | |
| | (mouse) | | | | | | |
| V$EGRF/NGFIC.01 | Nerve | 0.80 | 39-53 | (+) | 0.787 | 0.815 | agGCGGaggaggagg |
| | growth | | | | | | |
| | factor- | | | | | | |
| | induced | | | | | | |
| | protein C | | | | | | |
| V$MAZF/MAZ.01 | Myc | 0.90 | 46-58 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 52-66 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 64-76 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 69-85 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$EGRF/NGFIC.01 | Nerve | 0.80 | 81-95 | (+) | 0.768 | 0.801 | agGAGTaggaggaga |
| | growth | | | | | | |
| | factor- | | | | | | |
| | induced | | | | | | |
| | protein C | | | | | | |
| V$MAZF/MAZ.01 | Myc | 0.90 | 85-97 | (+) | 1.000 | 0.930 | gtagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 88-112 | (+) | 1.000 | 0.849 | ggaggAGAGagaggaggaggaggag |
| V$GABF/GAGA.01 | GAGA-Box | 0.78 | 90-114 | (+) | 1.000 | 0.792 | aggagAGAGaggaggaggaggagaa |
| V$RXRF/VDR_RXR.02 | VDR/RXR | 0.86 | 90-106 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 92-116 | (+) | 1.000 | 0.784 | gagagAGAGgaggaggaggagaaag |
| V$MAZF/MAZ.01 | Myc | 0.90 | 103-115 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 108-124 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 120-136 | (+) | 1.000 | 0.890 | aggaggtgAACAactta |
| | factor | | | | | | |
| | FKHRL1 | | | | | | |
| | (FOXO) | | | | | | |

Inspecting sequence WGS: humX (1-99):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 46-70 | (+) | 1.000 | 0.837 | gtaggAGAGagaggaggaggaggag |
| V$GABF/GAGA.01 | GAGA-Box | 0.78 | 48-72 | (+) | 1.000 | 0.792 | aggagAGAGaggaggaggaggagaa |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.784 | gagagAGAGgaggaggaggagaaag |
| V$MAZF/MAZ.01 | Myc | 0.90 | 61-73 | (+) | 1.000 | 0.909 | ggagGAGGagaaa |
| | associated | | | | | | |
| | zinc finger | | | | | | |
| | protein | | | | | | |
| | (MAZ) | | | | | | |
| V$BARB/BARBIE.01 | Barbiturate- | 0.88 | 67-81 | (+) | 1.000 | 0.882 | ggagAAAGaaggagg |
| | inducible | | | | | | |
| | element | | | | | | |
| V$GKLF/GKLF.01 | Gut- | 0.91 | 71-85 | (+) | 0.887 | 0.920 | aaagaaggagGAGGt |
| | enriched | | | | | | |
| | Krueppel- | | | | | | |
| | like factor | | | | | | |
| V$FKHD/FKHRL1.01 | Fkh-domain | 0.83 | 79-95 | (+) | 1.000 | 0.890 | aggaggtgAACAactta |
| | factor | | | | | | |
| | FKHRL1 | | | | | | |
| | (FOXO) | | | | | | |

Inspecting sequence WGS: humU (1-102):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 49-73 | (+) | 1.000 | 0.837 | gtaggAGAGagaggaggaggaggag |
| V$GABF/GAGA.01 | GAGA-Box | 0.78 | 51-75 | (+) | 1.000 | 0.792 | aggagAGAGaggaggaggaggagaa |
| V$RXRF/VDR_RXR.02 | VDR/RXR | 0.86 | 51-67 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 53-77 | (+) | 1.000 | 0.784 | gagagAGAGgaggaggaggagaaag |
| V$MAZF/MAZ.01 | Myc | 0.90 | 64-76 | (+) | 1.000 | 0.909 | ggagGAGGagaaa |
| | associated | | | | | | |
| | zinc finger | | | | | | |
| | protein | | | | | | |
| | (MAZ) | | | | | | |
| V$BARB/BARBIE.01 | Barbiturate- | 0.88 | 70-84 | (+) | 1.000 | 0.882 | ggagAAAGaaggagg |
| | inducible | | | | | | |
| | element | | | | | | |
| V$GKLF/GKLF.01 | Gut- | 0.91 | 74-88 | (+) | 0.887 | 0.920 | aaagaaggagGAGGt |
| | enriched | | | | | | |
| | Krueppel- | | | | | | |
| | like factor | | | | | | |
| V$FKHD/FKHRL1.01 | Fkh-domain | 0.83 | 82-98 | (+) | 1.000 | 0.890 | aggaggtgAACAactta |
| | factor | | | | | | |
| | FKHRL1 | | | | | | |
| | (FOXO) | | | | | | |

Inspecting sequence WGS: humY (1-102):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 49-73 | (+) | 1.000 | 0.837 | gtaggAGAGagaggaggaggaggag |
| V$GABF/GAGA.01 | GAGA-Box | 0.78 | 51-75 | (+) | 1.000 | 0.792 | aggagAGAGaggaggaggaggagaa |
| V$RXRF/VDR_RXR.02 | VDR/RXR | 0.86 | 51-67 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 53-77 | (+) | 1.000 | 0.784 | gagagAGAcgaggaggaggagaaag |
| V$MAZF/MAZ.01 | Myc | 0.90 | 64-76 | (+) | 1.000 | 0.909 | ggagGAGGagaaa |
| | associated | | | | | | |
| | zinc finger | | | | | | |
| | protein | | | | | | |
| | (MAZ) | | | | | | |
| V$BARB/BARBIE.01 | Barbiturate- | 0.88 | 70-84 | (+) | 1.000 | 0.882 | ggagAAAGaaggagg |
| | inducible | | | | | | |
| | element | | | | | | |
| V$GKLF/GKLF.01 | Gut- | 0.91 | 74-88 | (+) | 0.887 | 0.920 | aaagaaggagGAGGt |
| | enriched | | | | | | |
| | Krueppel- | | | | | | |
| | like factor | | | | | | |
| V$FKHD/FKHRL1.01 | Fkh-domain | 0.83 | 82-98 | (+) | 1.000 | 0.890 | aggaggtgAACAactta |
| | factor | | | | | | |
| | FKHRL1 | | | | | | |
| | (FOXO) | | | | | | |

Inspecting sequence Fem#5A1humX (1-98):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 12-28 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagaa |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.784 | gagagAGAGgaggaggaggagaaag |
| V$MAZF/MAZ.01 | Myc | 0.90 | 61-73 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 66-82 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 78-94 | (+) | 1.000 | 0.890 | aggaggtgAACAactta |
| | factor | | | | | | |
| | FKHRL1 | | | | | | |
| | (FOXO) | | | | | | |

Inspecting sequence WGS: chimpX (1-62):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$GKLF/GKLF.01 | Gut- | 0.91 | 13-27 | (+) | 0.887 | 0.920 | ggagaaagaaGAGGa |
| | enriched | | | | | | |
| | Krueppel- | | | | | | |
| | like factor | | | | | | |
| V$MAZF/MAZ.01 | Myc | 0.90 | 25-37 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 42-58 | (+) | 1.000 | 0.890 | aggaggtgAACAactta |
| | factor | | | | | | |
| | FKHRL1 | | | | | | |
| | (FOXO) | | | | | | |

Inspecting sequence Fem#5A2humX (1-98):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 12-28 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagaa |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.784 | gagagAGAGgaggaggaggagaaag |
| V$MAZF/MAZ.01 | Myc | 0.90 | 61-73 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 66-82 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 78-94 | (+) | 1.000 | 0.890 | aggaggtgAACAactta |
| | factor | | | | | | |
| | FKHRL1 | | | | | | |
| | (FOXO) | | | | | | |

Inspecting sequence Fem#3A1humX (1-134):

| | | | | | | | |
|---|---|---|---|---|---|---|---|
| V$MAZF/MAZ.01 | Myc | 0.90 | 7-19 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 12-28 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$RXRF/VDR_RXR.02 | VDR/RXR | 0.86 | 30-46 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$MAZF/MAZ.01 | Myc | 0.90 | 43-55 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 46-70 | (+) | 1.000 | 0.849 | ggaggAGAGagaggaggaggaggag |
| V$GABF/GAGA.01 | GAGA-Box | 0.78 | 48-72 | (+) | 1.000 | 0.792 | aggagAGAGaggaggaggaggagaa |
| V$RXRF/VDR_RXR.02 | VDR/RXR | 0.86 | 48-64 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 50-74 | (+) | 1.000 | 0.784 | gagagAGAGgaggaggaggagaaag |
| V$MAZF/MAZ.01 | Myc | 0.90 | 61-73 | (+) | 1.000 | 0.909 | ggagGAGGagaaa |
| | associated | | | | | | |
| | zinc finger | | | | | | |
| | protein | | | | | | |
| | (MAZ) | | | | | | |
| VRGABF/GAGA.01 | GAGA-Box | 0.78 | 70-94 | (+) | 0.750 | 0.831 | gaaagAGAAggaggaggagagagag |
| V$MAZF/MAZ.01 | Myc | 0.90 | 79-91 | (+) | 1.000 | 0.939 | ggagGAGGagaga |
| | associated | | | | | | |
| | zinc finger | | | | | | |
| | protein | | | | | | |
| | (MAZ) | | | | | | |
| V$GABF/GAGA.01 | GAGA-Box | 0.78 | 82-106 | (+) | 1.000 | 0.849 | ggaggAGAGagaggaggaggaggag |
| V$GABF/GAGA.01 | GAGA-Box | 0.78 | 84-108 | (+) | 1.000 | 0.792 | aggagAGAGaggaggaggaggagaa |
| V$RXRF/VDR_RXR.02 | VDR/RXR | 0.86 | 84-100 | (+) | 1.000 | 0.875 | aggagagaGAGGaggag |
| | Vitamin D | | | | | | |
| | receptor | | | | | | |
| | RXR | | | | | | |
| | heterodimer | | | | | | |
| | site | | | | | | |
| V$GABF/GAGA.01 | GAGA-Box | 0.78 | 86-110 | (+) | 1.000 | 0.784 | gagagAGAGgaggaggaggagaaag |
| V$MAZF/MAZ.01 | Myc | 0.90 | 97-109 | (+) | 1.000 | 0.909 | ggagGAGGagaaa |
| | associated | | | | | | |
| | zinc finger | | | | | | |
| | protein | | | | | | |
| | (MAZ) | | | | | | |
| V$RXRF/VDR_RXR.02 | VDR/RXR | 0.86 | 102-118 | (+) | 1.000 | 0.895 | aggagaaaGAGGaggag |
| | Vitamin D | | | | | | |
| | receptor | | | | | | |
| | RXR | | | | | | |
| | heterodimer | | | | | | |
| | site | | | | | | |
| V$FKHD/FKHRL1.01 | Fkh-domain | 0.83 | 114-130 | (+) | 1.000 | 0.890 | aggaggtgAACAactta |
| | factor | | | | | | |
| | FKHRL1 | | | | | | |
| | (FOXO) | | | | | | |

MatInspector (Quandt, K. et al)

TABLE 2

| | | | | | | | |
|---|---|---|---|---|---|---|---|
| | | | Position | | | | |
| | Further | | from- | | Core | Matrix | |
| Family/matrix | Information | Opt. | to | Str. | sim. | sim. | Sequence |

Inspecting sequence RefseqXY (1-65):

| | | | | | | | |
|---|---|---|---|---|---|---|---|
| V$VMYB/VMYB.05 | v-Myb, variant | 0.90 | 5-15 | (−) | 1.000 | 0.947 | aaaAACGgggg |
| | of AMV v-myb | | | | | | |
| V$GKLF/GKLF.01 | Gut-enriched | 0.91 | 6-20 | (−) | 0.852 | 0.930 | aacaaaaaaaCGGGg |
| | Krueppel-like | | | | | | |
| | factor | | | | | | |
| V$SORY/SRY.01 | Sex- | 0.95 | 7-23 | (−) | 1.000 | 0.951 | caaaACAAaaaaacggg |
| | determining | | | | | | |
| | region Y gene | | | | | | |
| | product | | | | | | |
| V$CABL/CABL.01 | Multifunctional | 0.97 | 12-22 | (−) | 1.000 | 0.997 | aaAACAaaaaa |
| | c-Abl src type | | | | | | |
| | tyrosine kinase | | | | | | |
| V$SORY/SRY.01 | Sex- | 0.95 | 12-28 | (−) | 1.000 | 0.950 | caaaACAAaacaaaaaa |
| | determining | | | | | | |
| | region Y gene | | | | | | |
| | product | | | | | | |
| V$SORY/SRY.01 | Sex- | 0.95 | 17-33 | (−) | 1.000 | 0.950 | caaaACAAaacaaaaca |
| | determining | | | | | | |
| | region Y gene | | | | | | |
| | product | | | | | | |
| V$SORY/SRY.01 | Sex- | 0.95 | 22-38 | (−) | 1.000 | 0.950 | caaaACAAaacaaaaca |
| | determining | | | | | | |
| | region Y gene | | | | | | |
| | product | | | | | | |
| V$SORY/SRY.01 | Sex- | 0.95 | 27-43 | (−) | 1.000 | 0.950 | caaaACAAaacaaaaca |
| | determining | | | | | | |
| | region Y gene | | | | | | |
| | product | | | | | | |
| V$SORY/SRY.01 | Sex- | 0.95 | 32-48 | (−) | 1.000 | 0.950 | caaaACAAaacaaaaca |
| | determining | | | | | | |
| | region Y gene | | | | | | |
| | product | | | | | | |

Inspecting sequence WGS: humX (1-65):

| | | | | | | | |
|---|---|---|---|---|---|---|---|
| V$VMYB/VMYB.05 | v-Myb, variant | 0.90 | 5-15 | (−) | 1.000 | 0.947 | aaaAACGgggg |
| | of AMV v-myb | | | | | | |
| V$GKLF/GKLF.01 | Gut-enriched | 0.91 | 6-20 | (−) | 0.852 | 0.930 | aacaaaaaaaCGGGg |
| | Krueppel-like | | | | | | |
| | factor | | | | | | |
| V$SORY/SRY.01 | Sex- | 0.95 | 7-23 | (−) | 1.000 | 0.951 | caaaACAAaaaaacggg |
| | determining | | | | | | |
| | region Y gene | | | | | | |
| | product | | | | | | |
| V$CABL/CABL.01 | Multifunctional | 0.97 | 12-22 | (−) | 1.000 | 0.997 | aaAACAaaaaa |
| | c-Abl src type | | | | | | |
| | tyrosine kinase | | | | | | |
| V$SORY/SRY.01 | Sex- | 0.95 | 12-28 | (−) | 1.000 | 0.950 | caaaACAAaacaaaaaa |
| | determining | | | | | | |
| | region Y gene | | | | | | |
| | product | | | | | | |
| V$GREF/GRE.01 | GLucocorticoid | 0.85 | 16-34 | (+) | 1.000 | 0.861 | ttgttttgttttGTTCtgt |
| | receptor, C2C2 | | | | | | |
| | zinc finger | | | | | | |
| | protein binds | | | | | | |
| | glucocorticoid | | | | | | |
| | dependent to | | | | | | |
| | GREs | | | | | | |
| V$SORY/SRY.01 | Sex- | 0.95 | 27-43 | (−) | 1.000 | 0.950 | caaaACAAaacagaaca |
| | determining | | | | | | |
| | region Y gene | | | | | | |
| | product | | | | | | |
| V$SORY/SRY.01 | Sex- | 0.95 | 32-48 | (−) | 1.000 | 0.950 | caaaACAAaacaaaaca |
| | determining | | | | | | |
| | region Y gene | | | | | | |
| | product | | | | | | |

Inspecting sequence WGS: humY (1-65):

| | | | | | | | |
|---|---|---|---|---|---|---|---|
| V$VMYB/VMYB.05 | v-Myb, | 0.90 | 5-15 | (−) | 1.000 | 0.947 | aaaAACGgggg |
| | variant of | | | | | | |
| | AMV v-myb | | | | | | |
| V$GKLF/GKLF.01 | Gut-enriched | 0.91 | 6-20 | (−) | 0.852 | 0.930 | aacaaaaaaaCGGGg |
| | Krueppel-like | | | | | | |
| | factor | | | | | | |
| V$AP1R/ | TCF11/MafG | 0.81 | 11-35 | (−) | 1.000 | 0.847 | aacagagcaTGACagaacaaaaaaa |
| TCF11MAFG.01 | heterodimers, | | | | | | |
| | binding to | | | | | | |
| | subclass of | | | | | | |
| | AP1 sites | | | | | | |
| V$GREF/PRE.01 | Progesterone | 0.84 | 21-39 | (+) | 1.000 | 0.900 | ctgtcatgctcTGTTctgt |
| | receptor | | | | | | |
| | binding site | | | | | | |

The sequence listing for each of the transcription factors is listed in Tables 1 and 2. The sequences can be supplied in the WIPO Standard ST25 if required.

The invention is not limited to the embodiments hereinbefore described which may be varied in detail.

### REFERENCES

Bakker S C, van der Meulen E M, Buitelaar J K, Sandkuijl L A, Pauls D L, Monsuur A J, van't Slot R, Minderaa R B, Gunning W B, Pearson P L, Sinke R J. A whole-genome scan in 164 Dutch sib pairs with attention-deficit/hyperactivity disorder: suggestive evidence for linkage on chromosomes 7p and 15q. Am J Hum Genet. 2003 May; 72(5):1251-60. Epub 2003 Apr. 04.
Barr C L, Feng Y, Wigg K, Bloom S, Roberts W, Malone M, Schachar R, Tannock R, Kennedy J L. Identification of DNA variants in the SNAP-25 gene and linkage study of these polymorphisms and attention-deficit hyperactivity disorder. Mol Psychiatry. 2000 July; 5(4):405-9.
Baron-Cohen S, Ring H A, Bullmore E T, Wheelwright S, Ashwin C, Williams S C. The amygdala theory of autism. Neurosci Biobehav Rev. 2000 May; 24(3):355-64.
Bolte E R, Autism and Clostridium tetani. Med Hypotheses. 1998 August; 51(2):133-44.
Brophy K, Hawi Z, Kirley A, Fitzgerald M, Gill M, Synaptosomal-associated protein 25 (SNAP-25) and attention deficit hyperactivity disorder (ADHD): evidence of linkage and association in the Irish population. Mol Psychiatry. 2002; 7(8):913-7.
Ciccodicola A et al. Differentially regulated and evolved genes in the fully sequenced Xq/Yq pseudoautosomal region. Hum Mol Genet. 2000 Feb. 12; 9(3):395-401.
CORPET F, Multiple sequence alignment with hierarchical clustering, 1988, Nucl. Acids Res., 16 (22), 10881-10890
Croonenberghs J, Bosmans E, Deboutte D, Kenis G, Maes M., Activation of the inflammatory response system in autism. Neuropsychobiology. 2002; 45(1): 1-6.
Croonenberghs J, Wauters A, Devreese K, Verkerk R, Scharpe S, Bosmans E, Egyed B, Deboutte D, Maes M., Increased serum albumin, gamma globulin, immunoglobulin IgG, and IgG2 and IgG4 in autism. Psychol Med. 2002 November; 32(8): 1457-63.
Fiona J S et al. Brief report: prevalence of autism spectrum conditions in children aged 5-11 years in Cambridgeshire, UK. Autism. 2002 September; 6(3):231-7.
Hallmayer J, Spiker D, Lotspeich L, McMahon W M, Petersen P B, Nicholas P, Pingree C, Ciaranello R D. Male-to-male transmission in extended pedigrees with multiple cases of autism. Am J Med Genet. 1996 Feb. 16; 67(1):13-8.
Heiner I, Eisfeld J, Luckhoff A, Role and regulation of TRP channels in neutrophil granulocytes. Cell Calcium. 2003 May-June; 33(5-6):533-40.
Hibino H, Pironkova R, Onwumere O, Vologodskaia M, Hudspeth A J, Lesage F, RIM binding proteins (RBPs) couple Rab3-interacting molecules (RIMs) to voltage-gated Ca(2+) channels. Neuron. 2002 Apr. 25; 34(3):411-23.
Jamain S, Quach H, Betancur C, Rastam M, Colineaux C, Gillberg I C, Soderstrom H, Giros B, Leboyer M, Gillberg C, Bourgeron T; Paris Autism Research International Sibpair Study. Mutations of the X-linked genes encoding neuroligins NLGN3 and NLGN4 are associated with autism. Nat. Genet. 2003 May; 34(1):27-9.
Jamain S, Quach H, Quintana-Murci L, Betancur C, Philippe A, Gillberg C, Sponheim E, Skjeldal O H, Fellous M, Leboyer M, Bourgeron T. Y chromosome haplogroups in autistic subjects. Mol Psychiatry. 2002; 7(2):217-9.
Levitt R C, McLane M P, MacDonald D, Ferrante V, Weiss C, Zhou T, Holroyd K J, Nicolaides N C, IL-9 pathway in asthma: new therapeutic targets for allergic inflammatory disorders. J Allergy Clin Immunol. 1999 May; 103(5 Pt 2):S485-91.
Li L, Hamer D H, Recombination and allelic association in the Xq/Yq homology region. Hum Mol Genet. 1995 November; 4(11):2013-6.
Liu J, Nyholt D R, Magnussen P, Parano E, Pavone P, Geschwind D, Lord C, Iversen P, Hoh J, Ott J, Gilliam T C; Autism Genetic Resource Exchange Consortium. A genomewide screen for autism susceptibility loci. Am J Hum Genet. 2001 August; 69(2):327-40.
Manning J T, Baron-Cohen S, Wheelwright S, Sanders G. The 2nd to 4th digit ratio and autism. Dev Med Child Neurol. 2001 March; 43(3):160-4.
Martinez-Arca S, Alberts P, Zahraoui A, Louvard D, Galli T. Role of tetanus neurotoxin insensitive vesicle-associated membrane protein (TI-VAMP) in vesicular transport mediating neurite outgrowth. J Cell Biol. 2000 May 15; 149(4):889-900.
Matarazzo M R, De Bonis M L, Gregory R I, Vacca M, Hansen R S, Mercadante G, D'Urso M, Feil R, D'Esposito M, Allelic inactivation of the pseudoautosomal gene SYBL1 is controlled by epigenetic mechanisms common to the X and Y chromosomes, Hum Mol Genet 2002 Dec. 1; 11(25):3191-8.
Newbury D F, Monaco A P. Molecular genetics of speech and language disorders. Curr Opin Pediatr. 2002 December; 14(6):696-701.
Nurmi E L, Bradford Y, Chen Y, Hall J, Arnone B, Gardiner M B, Hutcheson H B, Gilbert J R, Pericak-Vance M A, Copeland-Yates S A, Michaelis R C, Wassink T H, Santangelo S L, Sheffield V C, Piven J, Folstein S E, Haines J L, Sutcliffe J S. Linkage disequilibrium at the Angelman syndrome gene UBE3A in autism families. Genomics. 2001 September; 77(1-2):105-13.
O'Brien E K, Zhang X, Nishimura C, Tomblin J B, Murray J C. Association of specific language impairment (SLI) to the region of 7q31. Am J Hum Genet. 2003 June; 72(6): 1536-43.
Quandt, K. Frech, K. Karas, H. Wingender, E. and Werner, T. MatInd and MatInspector—New fast and versatile tools for detection of consensus matches in nucleotide sequence data Nucleic Acids Research 23, 4878-4884 (1995)
Petit E, Herault J, Raynaud M, Cherpi C, Perrot A, Barthelemy C, Lelord G, Muh J P. X chromosome and infantile autism. Biol Psychiatry. 1996 Sep. 15; 40(6):457-64.
Pickles A, Starr E, Kazak S, Bolton P, Papanikolaou K, Bailey A, Goodman R, Rutter M, Variable expression of the autism broader phenotype: findings from extended pedigrees. J Child Psychol Psychiatry. 2000 May; 41(4):491-502.
Schutz C K, Polley D, Robinson P D, Chalifoux M, Macciardi F, White B N, Holden J J. Autism and the X chromosome: no linkage to microsatellite loci detected using the affected sibling pair method. Am J Med Genet. 2002 Apr. 15; 109(1):36-41.
Senior K, Possible autoimmune enteropathy found in autistic children. Lancet. 2002 May 11; 359(9318):1674.
Shao Y, Wolpert C M, Raiford K L, Menold M M, Donnelly S L, Ravan S A, Bass M P, McClain C, von Wendt L, Vance J M, Abramson R H, Wright H H, Ashley-Koch A, Gilbert J R, DeLong R G, Cuccaro M L, Pericak-Vance M A Genomic screen and follow-up analysis for autisticdisorder. Am J Med Genet. 2002 Jan. 8; 114(1):99-105.
Singh V K, Plasma increase of interleukin-12 and interferon-gamma. Pathological significance in autism. J Neuroimmunol. 1996 May; 66(1-2):143-5.
Torrente F, Ashwood P, Day R, Machado N, Furlano R I, Anthony A, Davies S E, Wakefield A J, Thomson M A, Walker-Smith J A, Murch S H. Small intestinal enteropathy with epithelial IgG and complement deposition in children with regressive autism. Mol Psychiatry. 2002; 7(4):375-82, 334.
Wakefield A J, Murch S H, Anthony A, Linnell J, Casson D M, Malik M, Berelowitz M, Dhillon A P, Thomson M A, Harvey P, Valentine A, Davies S E, Walker-Smith J A, Ileal-lymphoid-nodular hyperplasia, non-specific colitis, and pervasive developmental disorder in children. Lancet. 1998 Feb. 28; 351(9103):637-41.

1. A method of screening for genetic or epigenetic markers associated with autism or related disorders comprising the steps of
isolating a biological sample from a mammal; and
testing the sample or genetic material isolated from the sample for genetic alterations.

2. A method as claimed in claim 1 wherein the genetic alterations comprise genetic polymorphisms or mutations and/or epigenetic alterations

3. A method as claimed in claim 2 wherein the polymorphism is located in the Xq/Yq pseudoautosomal gene region.

4. A method as claimed in claim 2 wherein the polymorphism is located in the Xq/Yq pseudoautosomal gene region and extends into the adjacent Xq28 gene region.

5. A method as claimed in claim 2 wherein the polymorphism is located in the Xq28 gene region adjacent to the Xq/Yq pseudoautosomal boundary.

6. A method as claimed in claim 2 wherein the polymorphism is located in the Yq region adjacent to the Xq/Yq pseudoautosomal boundary.

7. A method as claimed in claim 1 wherein the polymorphism is a deletion of variable length.

8. A method as claimed in claim 7 wherein the screening for deleted nucleic acids is carried out by a method selected from the group consisting of enzymatic cleavage and southern hybridisation; in situ hybridisation using probes from the specified region; detection of loss-of-heterozygosity using genetic analysis of polymorphic RFLP and microsatellite markers; and gene copy number analysis using real-time or other quantitative PCR technologies or DNA chip or array technologies.

9. A method as claimed in claim 2 wherein the polymorphism is selected from the group consisting of a chromosomal translocation, a chromosomal inversion, a gene conversion event, a reduction in gene dosage or gene expression of some or all of the genes that map to the specified region, an increase in gene dosage or gene expression of some or all of the genes that map to the specified region, an alteration in gene dosage or in the temporal or spatial aspects of gene expression of some or all of the genes that map to the specified region, an alteration in gene dosage or in the temporal or spatial aspects of gene expression of the HSPRY3 gene, and an alteration in gene dosage or in the temporal or spatial aspects of gene expression of the SYBL1 gene.

10. A method as claimed in claim 2 wherein the polymorphism involves a marker of epigenetic deregulation of gene expression.

11. A method as claimed in claim 2 wherein the genetic mutation is a deregulation of gene expression selected from the group consisting of an altered copy number or structure of DNA repeats in the HSPRY3 gene promoter, an alteration in the DNA sequence of the ‘MER31I c’ repeat in the HSPRY3 gene promoter, an alteration in the DNA sequence of the ‘GTTTT’ repeat downstream of the HSPRY3 gene transcriptional start site, an alteration of the DNA sequence downstream of the HSPRY3 gene protein coding region at the site of a recombination hotspot, and an alteration of the DNA sequence downstream of the HSPRY3 gene protein coding region at the site of a transcript expressed in the amygdala or other regions of the brain.

12. A method as claimed in claim 10 wherein the marker of epigenetic deregulation of gene expression is selected from the group consisting of an alteration in patterns of DNA methylation, an alteration in patterns of nuclease sensitivity of DNA or chromatin, an alteration in the protein composition of chromatin, loss-of-imprinting (reactivation) of the Y-linked copies of any one or more of the HSPRY3, SYBL1 and TRPC6-like genes, reactivation (biallelic expression) of the X-linked copies of any one or more of the HSPRY3, SYBL1 and TRPC6-like genes, silencing (transcriptional repression) of the X or Y linked copies of any one or more of the HSPRY3, SYBL1 and TRPC6-like genes, and increased or decreased mRNA or protein levels for the specified genes in the absence of detectable DNA sequence polymorphisms.

13. A method as claimed in claim 12 wherein the DNA sequence displaying abnormal levels of CpG methylation is the SYBL1 gene promoter-associated CpG island.

14. A method as claimed in claim 1 wherein the biological sample is selected from the group consisting of blood, saliva, semen, urine, amniotic fluid, placental biopsy, biopsy from a preimplantation stage embryo, biopsy from the chorionic villus (extraembryonic tissue) of an implanted embryo (fetus), fetal DNA or cells obtained from the serum of a pregnant mammal, hair, and tissue.

15. A method as claimed in claim 1 wherein the mammal is a human.

16. A method as claimed in claim 1 wherein the biological sample is isolated from developmentally disabled children or parents or relatives of developmentally disabled children.

17. A method of screening for genetic or epigenetic markers associated with autism and related disorders comprising the steps of:
isolating a biological sample from a mammal;
isolating the Xq/Yq pseudoautosomal region (PAR) region in the sample; and
comparing the isolated Xq/Yq pseudoautosomal region (PAR) region with a control sequence, wherein a deletion, addition or mutation indicates a susceptibility to autism or related disorders.

18. A method for screening for genetic or epigenetic markers associated with autism and related disorders comprising the steps of:
isolating a biological sample from a mammal;
isolating the HSPRY3 gene promoter region in the sample; and
comparing the isolated HSPRY3 region with a control sequence, wherein a deletion, addition or mutation indicates a susceptibility to autism or related disorders.

19. A method of screening for susceptibility to autism or related disorders comprising detecting an alteration in the HSPRY3 gene promoter region as listed in the group consisting of SEQ ID Nos 14, SEQ ID Nos 15, SEQ ID Nos 16, SEQ ID Nos 17 and SEQ ID Nos 18.

20. An antibody which specifically binds to an epitope of an altered marker encoded by genes in the Xq/Yq pseudoautosomal (PAR) region and adjacent chromosome-specific (Xq28) region.

21. An antibody which specifically binds to an epitope of an altered marker encoded by genes (listed in tables 1 and 2) that regulate genes in the Xq/Yq pseudoautosomal (PAR) region and adjacent chromosome-specific (Xq28) region.

22. An assay kit for screening for an alteration in the genetic or epigenetic markers associated with autism or related disorders comprising an antibody as claimed in claim 21 or a probe or primer selected from any one or more of SEQ ID No.s 1 to 13 and 35 to 41.

23. An assay kit as claimed in claim 22 comprising reagents suitable for western blot, immunohistochemical assays or ELISA assays.

24. An assay kit for screening for an alteration in the genetic or epigenetic markers associated with autism or related disorders comprising an antibody or probe or primer selected from the group consisting of SEQ ID Nos 1 to 13 and 35 to 41 which specifically binds to an epitope of an altered marker in the HSPRY3 gene promoter region.

25. An assay kit as claimed in claim 24 comprising reagents suitable for western blot, immunohistochemical assays or ELISA assays.

26. An assay kit for screening for an alteration in the genetic markers associated with autism or related disorders comprising an antibody or probe or primer that detects variants of the DNA, RNA or proteins associated the HSPRY3 or SYBL1 genes.

27. An assay kit as claimed in claim 26 comprising reagents suitable for western blot, immunohistochemical assays or ELISA assays.

28. An assay kit for screening for an alteration in the genetic markers associated with autism or related disorders comprising an antibody or probe or primer that detects variants of the DNA, RNA or proteins associated with genes that regulate expression of the HSPRY3 or SYBL1 genes.

29. An assay kit as claimed in claim 28 comprising reagents suitable for western blot, immunohistochemical assays or ELISA assays.

30. A DNA sequence comprising a nucleic acid sequence selected from the group consisting of SEQ ID Nos. 1 to 13 and SEQ ID Nos. 35 to 41.

31. A DNA sequence comprising a nucleic acid sequence selected from the group consisting of Seq ID Nos. 14 to 18 and Seq ID Nos. 27 to 34.

32. A method for the treatment of autism and/or related disorders in patients having genetic markers associated with autism or related disorders comprising detecting in a biological sample genetic polymorphisms/mutations and/or epigenetic alterations in the Xq/Yq pseudoautosomal gene region and providing appropriate treatment.

33. A method as claimed in claim 32 wherein the treatment comprises a pharmaceutically acceptable active agent for administration based on the polymorphisms/mutations and/or epigenetic alterations.

34. A method for the treatment of autism and/or related disorders in patients having genetic markers associated with autism or related disorders comprising the steps of:—
detecting in a biological sample genetic polymorphisms/mutations and/or epigenetic alterations in the Xq/Yq pseudoautosomal gene region; and
providing treatment in the form of any one or more of
early behaviour training; or
early dietary interventions or manipulations.

35. A method for the treatment and/or prophylaxis of autism and/or related disorders in patients having genetic or epigenetic markers associated with autism or related disorders comprising the steps of:—
detecting in a biological sample genetic polymorphisms/mutations and/or epigenetic alteration in the Xq/Yq pseudoautosomal gene region; and
providing any one or more of
gene therapy;
activation or reactivation of epigenetically silenced genes; or
silencing or reducing gene expression at the mRNA or protein level.

36. A method for the treatment and/or prophylaxis of autism and/or related disorders in patients having genetic or epigenetic markers associated with autism or related disorders comprising the steps of:—
detecting in a biological sample genetic polymorphisms/mutations and/or epigenetic alteration in the Xq/Yq pseudoautosomal gene region; and
providing a pharmaceutically acceptable active agent for administration wherein epigenetically silenced genes are activated or reactivated; or wherein gene expression at the mRNA or protein level are silenced or reduced.

37. A method as claimed in claim 35 wherein the polymorphism is located in any one or more of the Xq/Yq pseudoautosomal gene region and extends into the adjacent Xq28 gene region, the Xq28 gene region adjacent to the Xq/Yq pseudoautosomal boundary, the HSPRY3 gene promoter region, the SYBL1 gene

38. A method for the treatment and/or prophylaxis of autism and/or related disorders in children comprising identifying genetic markers associated with autism or related disorders.

39. A method for the treatment and/or prophylaxis of autism and/or related disorders in children comprising activation or reactivation of epigenetically silenced genes in the Xq/Yq pseudoautosomal gene region.

40. A method for the treatment and/or prophylaxis of autism and/or related disorders in children comprising the step of silencing or reducing gene expression at the mRNA or protein level in the Xq/Yq pseudoautosomal gene region.

41. A method for selectively inhibiting HSPRY3, AMD2; SYBL1, TRPC6-like, IL9R or CXYorf1 activity in a human host, comprising administering a compound which selectively inhibits the activity of the gene products of any one or more of HSPRY3, AMD2, SYBL1, TRPC6-like, IL9R and CXYorf1.

42. A method for selectively enhancing or inhibiting the activity of proteins that regulate the HSPRY3 or SYBL1 genes (Tables 1 and 2) in a human host, comprising administering a compound which selectively enhances or inhibits the activity of the gene products selected from the group consisting of genes listed in tables 1 and 2.

43. A method for the treatment and/or prophylaxis of tetanus susceptibility, tuberous sclerosis (TS) or attention deficit/hyperactivity disorder (AD/HD) in patients comprising identifying genetic or epigenetic markers associated with autism.

44. A method for the treatment and/or prophylaxis of tetanus susceptibility, tuberous sclerosis (TS) or attention deficit/hyperactivity disorder (AD/HD) in patients comprising activation or reactivation of epigenetically silenced genes in the Xq/Yq pseudoautosomal gene region.

45. A method for the treatment and/or prophylaxis of tetanus susceptibility, tuberous sclerosis (TS) or attention deficit/hyperactivity disorder (AD/HD) in patients comprising the step of silencing or reducing gene expression at the mRNA or protein level in the Xq/Yq pseudoautosomal gene region.

46. A method of assessing the personality of a patient or their susceptibility to autism or related disorders comprising the step of genotyping the ASD locus comprising genes in the Xq/Yq PAR region.

47. A vector suitable for gene therapy comprising one or more of the genes in the Xq/Yq pseudoautosomal region (PAR) and adjacent X chromosome-specific (Xq28) region.

48. A vector suitable for gene therapy comprising the HSPRY3 gene promoter region of the HSPRY3 gene (Accession No. AJ271735).

49. A vector suitable for gene therapy comprising the SYBL1 gene (Accession No. AJ271736).
